(19) United States
(12) Patent Application Publication
(10) Pub. No.: US 2024/0238107 A1
(43) Pub. Date: Jul. 18, 2024

(54) SYSTEMS AND METHODS FOR CONTROLLING A ROBOTIC ARM BASED ON BRAIN ACTIVITIES

(71) Applicant: Imam Abdulrahman Bin Faisal University, Dammam (SA)

(72) Inventors: Murad Mohsen ALTHOBAITI, Dammam (SA); Tasneem Mahmoud AHMED, Dammam (SA); Fatemah Tawfiq ALSAHAF, Dammam (SA); Reem Nabeel ALOMAIR, Dammam (SA); Renad Ali ALNAMLAH, Dammam (SA); Reem Abdulrabalameer ALGHAFLI, Dammam (SA)

(73) Assignee: Imam Abdulrahman Bin Faisal University, Dammam (SA)

(21) Appl. No.: 18/154,083

(22) Filed: Jan. 13, 2023

(51) Int. Cl. A61F 2/54; A61B 5/00; A61F 2/72

(52) U.S. Cl. CPC A61F 2/54; A61B 5/4064; A61F 2/72

(57) ABSTRACT

A method of controlling robotic arm based on brain activities. The method includes measuring HbO level of non-disabled subject at target brain areas during wrist movement using a fNIRS device with light sources and detectors. The method further includes detecting brain activities of non-disabled subject through said detectors based on HbO level of non-disabled subject during wrist movement, and classifying brain activities corresponding to wrist movement using classification algorithms and generating training data set. The method also includes generating control signals based on brain activities for robotic arm to perform wrist movement, and detecting brain activities of disabled subject at target brain areas based on HbO level using fNIRS device. The method includes analyzing brain activities of disabled subject based on training data set, and generating control signal for robotic arm to perform wrist movement based on analyzed brain activity of disabled subject.

### BACKGROUND

### Technical Field

The present disclosure is directed to systems and methods for controlling a robotic arm based on brain activities.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Amputation is the removal of a limb such as arm, leg, foot, hand, toe, or finger by trauma, medical illness, or surgery. Despite advances in medicine and surgery, amputation continues to be a significant problem. The amputation problem has been recognized as a global issue involving social, economic, industrial, and psychological issues that demand assistance and advice. Therefore, it is critical to understand the ways that have been or could be developed to reduce the inconvenience caused by such injuries through adequate rehabilitation. The number of patients diagnosed with neuromuscular disorders has increased significantly in recent years. As a result, researchers have focused on evaluating brain activity during motor task execution in recent years. Many brain imaging techniques, such as functional magnetic resonance imaging (fMRI), single-photon emission computed tomography (SPECT), and magnetoencephalography (MEG) may be used to obtain practical information about the brain. These techniques, however, have several drawbacks, such as immobility, cost, and motion artifacts.

Accordingly, it is one object of the present disclosure to evaluate brain activity during motor task execution in a cost effective and in an efficient manner.

### SUMMARY

In an exemplary embodiment, a method of controlling a robotic arm based on brain activities is disclosed. The method includes measuring a hyperbaric oxygen (HbO) level of a non-disabled subject at target brain areas during a wrist movement using functional near-infrared spectroscopy (fNIRS) device with light sources and detectors. The method further includes detecting one or more brain activities of the non-disabled subject through said detectors based on the HbO level of the non-disabled subject during the wrist movement. Further, the method includes classifying the one or more brain activities corresponding to the wrist movement using one or more classification algorithms and generating a training data set. The method includes generating one or more control signals based on the one or more brain activities for the robotic arm to perform the wrist movement. The method includes detecting one or more brain activities of a disabled subject at the target brain areas based on the HbO level using the fNIRS device. The method also includes analyzing the one or more brain activities of the disabled subject based on the training data set. Further, the method includes generating the one or more control signal for the robotic arm to perform the wrist movement based on the analyzed brain activity of the disabled subject.

In another exemplary embodiment, a robot arm is disclosed. The robot arm includes a joint pin to connect a pin finger, a first finger, and a proximal finger together in a palm section of the robot arm, wherein the palm section further comprises a palm. The robot arm further includes a wrist connector and a hand connector to connect the palm section to a forearm section through a wrist joint. The forearm section comprises an actuator base mounted on a circuit holder to control a movement of the robot arm.

In yet another exemplary embodiment, a system of provisioning control of a robotic arm based on brain activities is disclosed. The system includes a robotic arm, a functional near-infrared spectroscopy (fNIRS) device with one or more light sources and one or more detectors for measuring a hyperbaric oxygen (HbO) level of at least one non-disabled subject at target brain areas during a wrist movement, where the one or more detectors detects one or more brain activities of the non-disabled subject based on the HbO level of the non-disabled subject during the wrist movement. The system further includes a classifying means for classifying the one or more brain activities corresponding to wrist movement using one or more classification algorithms and generating a training data set. The system includes a brain-control interface (BCI) that generates one or more control signals based on the classified brain activities for the robotic arm to perform the wrist movement. The system further includes a detecting means for detecting one or more brain activities of a disabled subject at the target brain areas based on the HbO level using the fNIRS device. The system also includes an analyzing means for analyzing the one or more brain activities of the disabled subject based on the training data set, where the BCI generates the control signal for the robotic arm to perform the wrist movement based on the analyzed brain activity of the disabled subject.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 depicts a graphical plot illustrating schematic of absorbance spectrum of oxygenated hemoglobin and deoxygenated hemoglobin used by functional near-infrared spectroscopy (fNIRS) technology, according to aspects of the present disclosure;

FIG. 2 depicts a fNIRS-brain-control interface (BCI) schematic, according to aspects of the present disclosure;

FIG. 3 depicts a system for provisioning control of a robotic arm based on brain activities, according to aspects of the present disclosure;

FIG. 4 depicts a robotic arm, according to aspects of the present disclosure;

FIG. 4B depicts a feedback device, according to aspects of the present disclosure;

FIG. 5 depicts an electrical circuit, according to aspects of the present disclosure;

FIG. 6A to FIG. 6N depict different parts of upper limb prototype, according to aspects of the present disclosure;

FIG. 7 depicts an optodes arrangement, according to aspects of the present disclosure;

FIG. 8 depicts another optodes arrangement, according to aspects of the present disclosure;

FIG. 9A illustrates an experimental paradigm of a real experiment, according to aspects of the present disclosure;

FIG. 9B illustrates an experimental paradigm of an imagery experiment, according to aspects of the present disclosure;

FIG. 10A illustrates an example of individual subject maps of hyperbaric oxygen (HbO) changes during real experiment, according to aspects of the present disclosure;

FIG. 10B illustrates an example of individual subject maps of HbO changes during imagery experiment, according to aspects of the present disclosure;

FIGS. 11A, 11B, 11C, and 11D show a hemodynamic response corresponding to real wrist tasks, according to aspects of the present disclosure;

FIGS. 12A, 12B, 12C, and 12D show a hemodynamic response corresponding to imagery wrist tasks, according to aspects of the present disclosure;

FIG. 13 depicts a real wrist tasks confusion matrix, according to aspects of the present disclosure;

FIG. 14 depicts imagery wrist tasks confusion matrix, according to aspects of the present disclosure;

FIG. 15 shows a digital prototype, according to aspects of the present disclosure;

FIG. 16 shows a real prototype, according to aspects of the present disclosure;

FIG. 17 shows a confusion matrix of a predicted class, according to aspects of the present disclosure;

FIG. 18 describes wrist movement development, according to aspects of the present disclosure;

FIG. 19 depicts NIRStar15-2 software interface, according to aspects of the present disclosure;

FIG. 20 depicts nirsLAB software interface, according to aspects of the present disclosure;

FIG. 21 depicts classification learner application interface, according to aspects of the present disclosure;

FIG. 22 illustrates a method for controlling a robotic arm based on brain activities, according to aspects of the present disclosure;

FIG. 23 is an illustration of a non-limiting example of details of computing hardware used in the computing system, according to aspects of the present disclosure;

FIG. 24 is an exemplary schematic diagram of a data processing system used within the computing system, according to aspects of the present disclosure;

FIG. 25 is an exemplary schematic diagram of a processor used with the computing system, according to aspects of the present disclosure; and

FIG. 26 is an illustration of a non-limiting example of distributed components which may share processing with the controller, according to aspects of the present disclosure.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of the present disclosure are directed to systems and methods for development of a robotic arm based on functional near-infrared spectroscopy (fNIRS)-brain-control interface (BCI) for rehabilitation protocols improvement. The systems and methods may be configured to control the robotic arm based on brain activities. The present disclosure employs fNIRS technology to control the robotic arm based on brain activities.

BCI promotes locked-in people to operate machines by reading neural impulses directly from the brain, obviating the need for peripheral nerve and muscle input. An emerging technology in the BCI is the fNIRS technology that uses light to detect brain activations linked to the motor activities of the people.

The fNIRS technology can measure brain activation level corresponding to body movements, including hand movements. The use of fNIRS technology aids in the recovery and restoration of control over hand movements. In examples, the fNIRS technology may be used to obtain hemodynamic response signals of limb movements such as the four primary wrist movements during both imagery and execution tasks.

The fNIRS technology is based on assessing variations in cerebral blood flow hemodynamics, i.e., variations in oxygenated and deoxygenated hemoglobin concentrations. NI lights with multiple emitters and detector sets that operate at two or many wavelengths are used in fNIRS. Simultaneous photon diffraction causes the light released into the scalp to distribute into the brain tissues. With carefully positioned detectors, the outgoing photons are measured, and the intensity levels of the observed light is used to calculate changes in oxygenated hemoglobin and deoxygenated hemoglobin concentrations along the photon route. Because of its numerous benefits, fNIRS technique may be applied in BCI applications. fNIRS is preferable due to its ease of use, portability, safety, low noise, and lack of sensitivity to electrical noise.

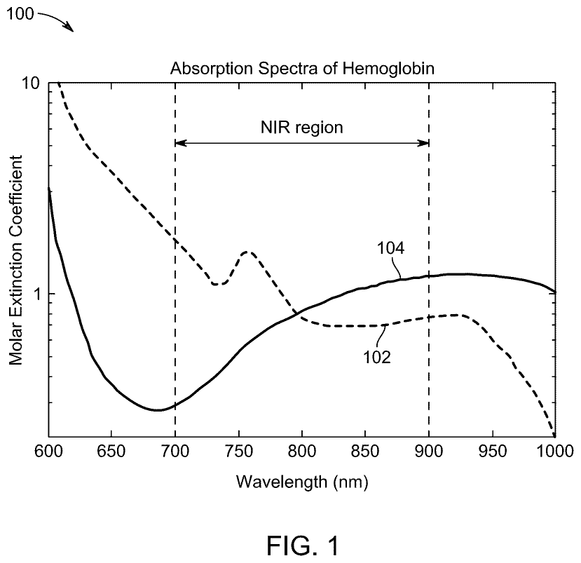

FIG. 1 depicts a graphical plot 100 illustrating schematic of absorbance spectrum of HbO and HbR used by the fNIRS technology, according to aspects of the present disclosure.

In FIG. 1, plot line 102 represents absorption spectrum of deoxygenated hemoglobin and plot line 104 represents absorption spectrum of oxygenated hemoglobin. FIG. 1 shows the manner in which fNIRS system monitors hemodynamic responses by transmitting Near-Infrared (NIR) light into the cortex and receiving the reflected light. Hemoglobin and deoxygenated hemoglobin can be determined based on absorption spectra of the oxygenated hemoglobin and deoxygenated hemoglobin measured at the detectors.

During amputation, it is critical to condition, trim, and smooth the remaining bone so that a healthy stump can bear a load of a prosthetic limb in the future and limit the risk of complications. The muscle is sutured to the bone at the distal residual bone to maintain maximum strength in the remaining limb. A prosthesis, or prosthetic device, can assist in rehabilitation. For many patients, an artificial limb can improve mobility and ability to manage daily tasks while also allowing them to remain self-sufficient. Advances in the field of bio mechatronics have opened new avenues for amputees to use and apply prosthetic devices. The control of such prosthetic limbs, on the other hand, is a new topic for researchers to investigate. Intention detection through bio signals is preferred, which then prompts control implementation. The human hand contains a complicated mechanism that allows it to execute useful tasks. The wrist and hand have a wide range of motion, which is important for gripping and interacting with items. Abduction, adduction, extension, and flexion are all wrist movements. As a result, there is a need for a specific prosthetic that uses fNIRS to aid the patient meet his/her needs.

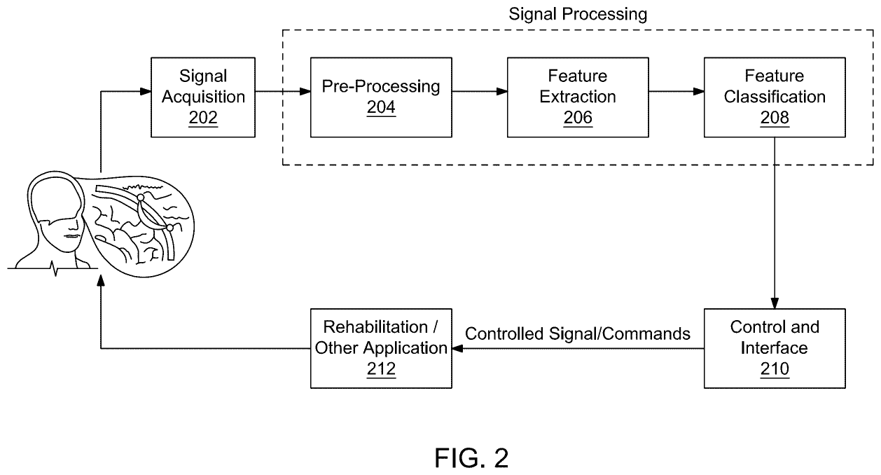

FIG. 2 depicts a fNIRS-BCI schematic, according to aspects of the present disclosure. As described in FIG. 2, the fNIRS-BCI schematic includes multiple stages including a signal acquisition stage 202, a pre-processing stage 204, a feature extraction stage 206, a feature classification stage 208, a control and interface stage 210, and a rehabilitation stage 212. During the signal acquisition stage 202, suitable brain signals may be gathered using an appropriate brain-imaging modality. The obtained brain signals are typically frail and carry disturbances (for example, mechanical and physiological disturbances) and artifacts. Further, during the pre-processing stage 204, the obtained brain signals are processed. During the feature extraction stage 206, features are extracted from the processed brain signals. Further, during the feature classification stage 208, the extracted features are classified by employing a classifier. Finally, the classified features are sent to other outer instruments in the control and interface stage 210 to generate the required control commands for a robotic arm in a form of rehabilitation.

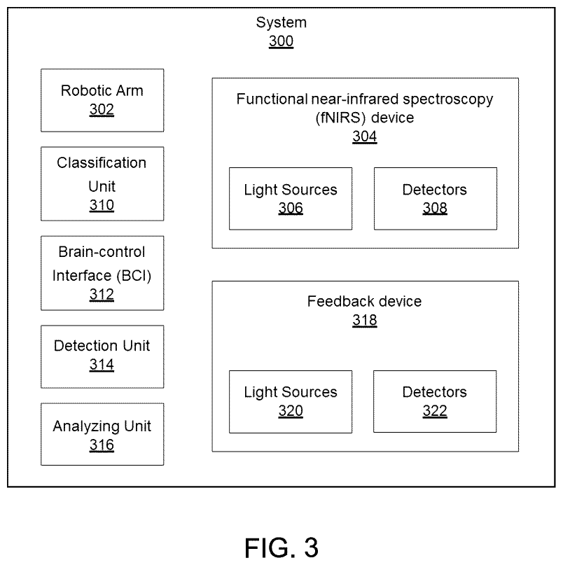

FIG. 3 depicts a system 300 for provisioning control of a robotic arm based on brain activities, according to aspects of the present disclosure.

The system 300 may include a robotic arm 302 (interchangeably referred to as robot arm 302). The system 300 also includes a fNIRS device 304. The fNIRS device 304 includes one or more light sources 306 and one or more detectors 308. In an example, the one or more light sources 306 may emit light at a first peak emission of 760±10 nm and at a second peak emission of 850±10 nm. In examples, the system 300 may include eight light sources and eight detectors. Further, the system 300 includes a classification unit 310, a BCI 312, a detection unit 314, and an analyzing unit 316. The system 300 may also include a feedback device 318 to indicate the correctness of fNIRS signals compare to the wrist movement. The feedback device 318 includes one or more light sources 320 and one or more detectors 322.

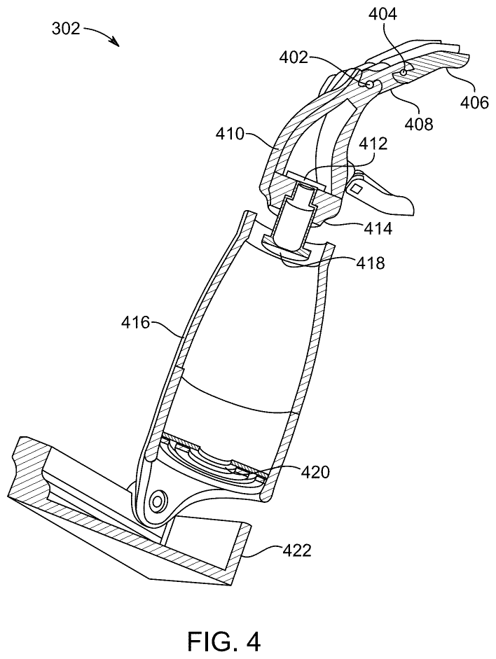

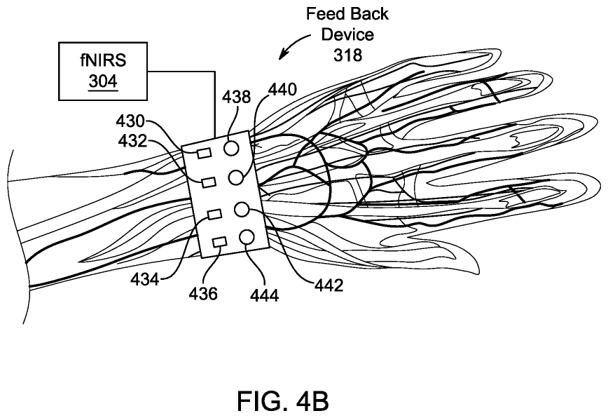

FIG. 4 depicts the robotic arm 302, according to aspects of the present disclosure. The robotic arm 302 includes a joint pin 402, a pin finger 404, a first finger 406, a proximal finger 408, a palm section 410, a wrist connector 412, a hand connector 414, a forearm section 416, a wrist joint 418, an actuator base 420, and a circuit holder 422. The joint pin 402 connects the pin finger 404, the first finger 406, and the proximal finger 408 together in the palm section 410 of the robotic arm 302. The palm section 410 comprises a palm. Further, the wrist connector 412 and the hand connector 414 connect the palm section 410 to the forearm section 416 through the wrist joint 418. The forearm section 416 comprises the actuator base 420 mounted on the circuit holder 422 to control a movement of the robotic arm 302. According to aspects of the present disclosure, the robotic arm 302 is configured to perform four wrist movements including wrist extension, wrist flexion, ulnar deviation, and radial deviation.

Referring back to FIG. 3, the fNIRS device 304 may be configured to measure a hyperbaric oxygen (HbO) level of at least one non-disabled subject at target brain areas during a wrist movement. The wrist movement includes at least one selected from the group consisting of wrist extension, wrist flexion, ulnar deviation, and radial deviation of the non-disabled subject. In an aspect, the one or more detectors 308 may be configured to detect one or more brain activities of the non-disabled subject based on the HbO level of the non-disabled subject during the wrist movement.

In an aspect, the classification unit 310 may be a classifying means for classifying the one or more brain activities corresponding to wrist movement using one or more classification algorithms and generating a training data set. The one or more classification algorithms include Artificial Neural Networks (ANN), K-Nearest Neighbor (KNN) or Support Vector Machine (SVM).

The BCI 312 may be a direct communication pathway between the brain activities and the robotic arm 302. In an aspect, the BCI 312 may be configured to generate one or more control signals based on the classified brain activities for the robotic arm 302 to perform the wrist movement.

The detection unit 314 may be a detecting means for detecting one or more brain activities of a disabled subject at the target brain areas based on the HbO level using the fNIRS device 304. The analyzing unit 316 may be an analyzing means for analyzing the one or more brain activities of the disabled subject based on the training data set. In an aspect, the BCI 312 may generate the control signal for the robotic arm 302 to perform the wrist movement based on the analyzed brain activity of the disabled subject.

FIG. 4B depicts the feedback device 318, according to the aspects of the present disclosure. The feedback device 318 includes four LED light sources 430, 432, 434, 436 and four detectors 438, 440, 442, 444. The feedback device 318 is connected to the fNIRS device 304. The feedback device 318 detects the actual movement of the wrist via the four detectors 430, 432, 434, 436 and compares the reading with the classified wrist movement from the classification unit 310 and lights up the four LED light sources 438, 440, 442, 444 according to the comparison.

In a preferred embodiment of the invention the feedback device 318 acts as a training reinforcer for the subject. Lights present on the optode arrangement of particular wavelength/color and/or intensity corresponds with one or more lights disposed on the feedback device 318 when mounted on the wrist of the subject. Lights corresponding with particular brain activity are duplicated on the Feedback device 318. In this way a subject obtains reinforcing training to more quickly train the algorithm and/or the subject to recognize and correlate particular brainwave activity with a particular wrist movement (motor control). The feedback device 318 is preferably in the form of a plurality of links that snuggly contact the skin surface of the subject's wrist. In this form the links are similar to the links of a watch wristband. The number of links may vary from 8-64 with lights regularly or irregularly spaced among links (see for example FIG. 4B). A wireless connection between the optode arrangement and the feedback device 318 permits activation of same-wavelength lights on the feedback device 318 at an intensity that is proportional to the intensity observed on the optode arrangement.

The following examples are provided to illustrate further and to facilitate the understanding of the present disclosure.

An experiment was conducted to evaluate fNIRS ability to detect a hemodynamic response in the primary, secondary, and premotor cortices areas when adult subjects were asked to perform four wrist movements, namely wrist extension, wrist flexion, ulnar deviation, and radial deviation. In particular, the hemodynamic response resulting from brain activation due to the execution of four wrist movements was investigated to prove that a significant difference can be observed using fNIRS technology.

Twelve healthy subjects with no history of neurological or mental condition participated in the experiment (mean age of 20±3 years, seven female subjects, and five male subjects). Only right-handed volunteers were selected to rule out any discrepancies in hemodynamic responses related to hemisphere dominance. All the subjects had healthy or corrected-to-normal eyesight, and after being fully briefed about the experimental process, they all gave their verbal consent. Information about subjects is described in Table 1 provided below.

TABLE 1
Information about subjects (participants)

| ID | Gender | Age | Dominant Hand |
|---|---|---|---|
| 01 | Female | 23 | Right |
| 02 | Female | 22 | Right |
| 03 | Female | 22 | Right |
| 04 | Female | 23 | Right |
| 05 | Female | 23 | Right |
| 06 | Male | 22 | Right |
| 07 | Female | 22 | Right |
| 08 | Female | 23 | Right |
| 09 | Male | 20 | Right |
| 10 | Male | 21 | Right |
| 11 | Male | 21 | Right |
| 12 | Male | 21 | Right |

The hemodynamic data was exported from the nirsLAB software and was further imported into the MATLAB program. The hemodynamic data were processed to extract features. The extracted features were used to predict the wrist movement by a classification model (explained later). After the MATLAB program processed the hemodynamic data and predicts the wrist movement, the output of the MATLAB program was used as conditions for an Arduino microcontroller. In examples, the output of the MATLAB program is in a numerical form, for example, “1,” “2”, “3”, or “4,” corresponding to the four movements of the wrist including wrist extension, wrist flexion, ulnar deviation, and radial deviation, respectively. In order to obtain the desired output, an electrical circuit was implemented.

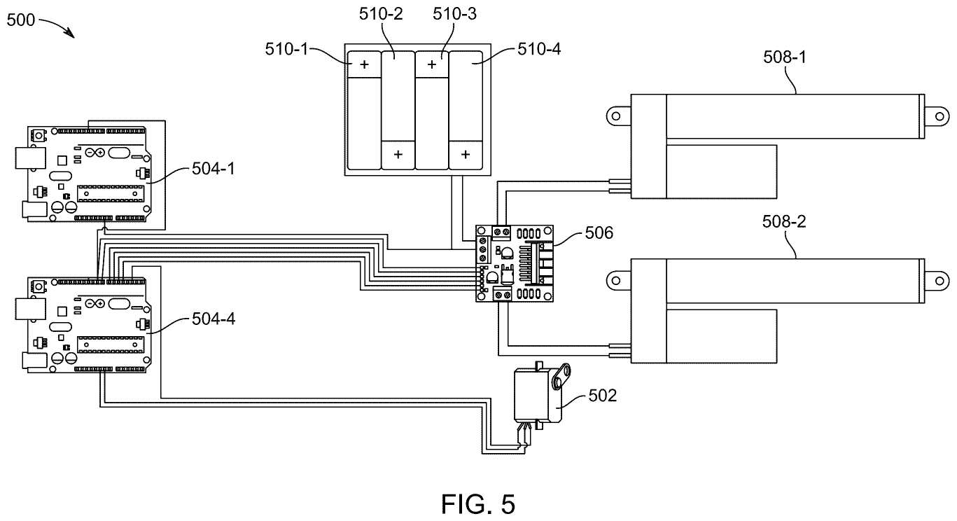

FIG. 5 depicts the electrical circuit 500, according to aspects of the present disclosure. The electrical circuit 500 includes a servo motor 502 and two Arduino microcontrollers, i.e., a first Arduino microcontroller 504-1 and a second Arduino microcontroller 504-2. The electrical circuit 500 further includes a motor driver 506 and two linear actuators, i.e., a first linear actuator 508-1 and a second linear actuator 508-2. The electrical circuit 500 further includes four batteries, i.e., a first battery 510-1, a second battery 510-2, a third battery 510-3, and a fourth battery 510-4. The motor driver 506 is an L298N motor driver. The first Arduino microcontroller 504-1 and the second Arduino microcontroller 504-2 are connected to the motor driver 506 driving the first linear actuator 508-1 and the second linear actuator 508-2 powered by the first battery 510-1, the second battery 510-2, the third battery 510-3, and the fourth battery 510-4. The servo motor 502 is an MG996R servo motor. The servo motor 502 provides 180 degrees rotation and will be attached to a base that will rotate 90 degrees back and forth. It is shock-proof, accurate, does not need a feedback loop, and has a stall torque of 9.4-11 kgf·cm depending on the voltage supplied, making it suitable to obtain the desired accurate fixed movement. The first Arduino microcontroller 504-1 and the second Arduino microcontroller 504-2 are Arduino UNO R3 microcontrollers. The first Arduino microcontroller 504-1 and the second Arduino microcontroller 504-2 are used to get input (code) from the MATLAB program, store the code, and run the stored code. The first Arduino microcontroller 504-1 and the second Arduino microcontroller 504-2 also power the components of the electrical circuit 500 by +5V and the GND pins.

The first linear actuator 508-1 and the second linear actuator 508-2 may move loads in a straight path. The first linear actuator 508-1 and the second linear actuator 508-2 alert the motor rotational motion to a linear motion allowing forward and backward movements. Their push and pull movements enable lifting the devices attached to them as well as tipping and sliding them. The first linear actuator 508-1 and the second linear actuator 508-2 are used to control the wrist movement based on their length.

The maximum voltages the first Arduino microcontroller 504-1 and the second Arduino microcontroller 504-2 can output are 3.3V and 5V. However, the first linear actuator 508-1 and the second linear actuator 508-2 operate at voltages of at least 5V to 12V. In addition, the limit current that the first Arduino microcontroller 504-1 and the second Arduino microcontroller 504-2 can provide is very low compared to the need of the servo motor 502 and the motor driver 506. As a result, the first Arduino microcontroller 504-1 and the second Arduino microcontroller 504-2 alone are insufficient to power up the servo motor 502 and the motor driver 506. Consequently, the use of the motor driver 506 to step up the output from the first Arduino microcontroller 504-1 and the second Arduino microcontroller 504-2 to fill the gap between the first Arduino microcontroller 504-1 and the second Arduino microcontroller 504-2, the first linear actuator 508-1 and the second linear actuator 508-2 are essential. The IN1 & IN2 pins control the first linear actuator 508-1, and IN3 & IN4 pins control the second linear actuator 508-2. In examples, the logic table of linear actuator control system is illustrated in Table 2 provided below.

TABLE 2
Logic table of linear actuator control system

| IN1 | IN2 | Result | IN1 | IN2 | Result |
|---|---|---|---|---|---|
| 1 | 0 | First linear actuator | 1 | 0 | Second linear actuator |
| | | moves up | | | moves up |
| 0 | 1 | First linear actuator | 0 | 1 | Second linear actuator |
| | | moves down | | | moves down |
| 1 | 1 | First linear actuator | 1 | 1 | Second linear actuator |
| | | Stops | | | stops |
| 0 | 0 | First linear actuator | 0 | 0 | Second linear actuator |
| | | Stops | | | stops |

The first Arduino microcontroller 504-1 is used as a link between the output of the MATLAB program and the electrical circuit 500. The output is in numerical form of either 1, 2, 3, or 4 and will be stored in Pin 11 of the first Arduino microcontroller 504-1, and will be used as an input to the second Arduino microcontroller 504-2. Six pins of the second Arduino microcontroller 504-2 are connected to the motor driver 506 to control the speed and direction of the servo motor 502 and the motor driver 506. Finally, the servo motor 502 is used to rotate the base on which the first linear actuator 508-1 and the second linear actuator 508-2 will be fixed to change the axis of motion.

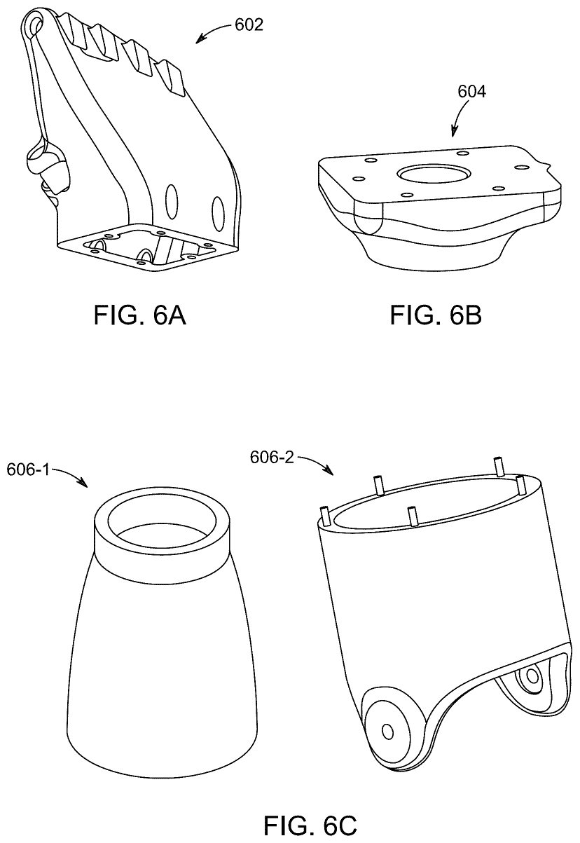

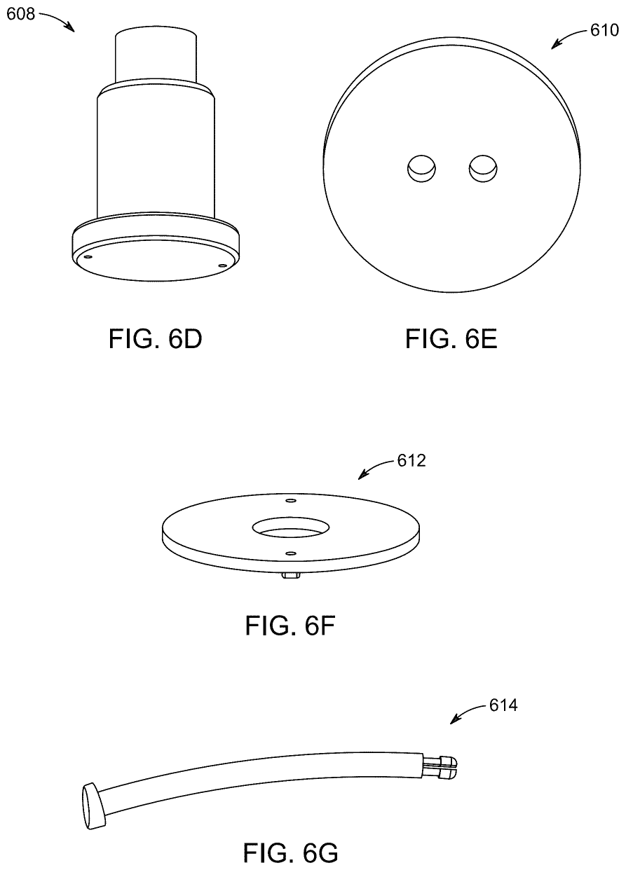

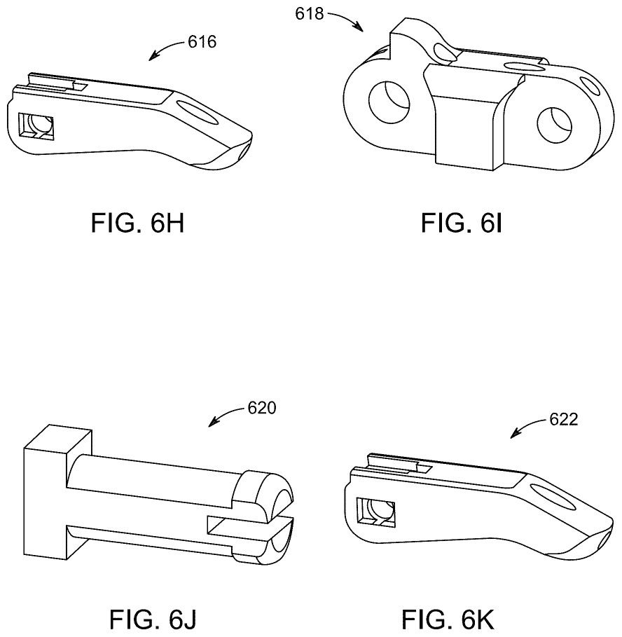

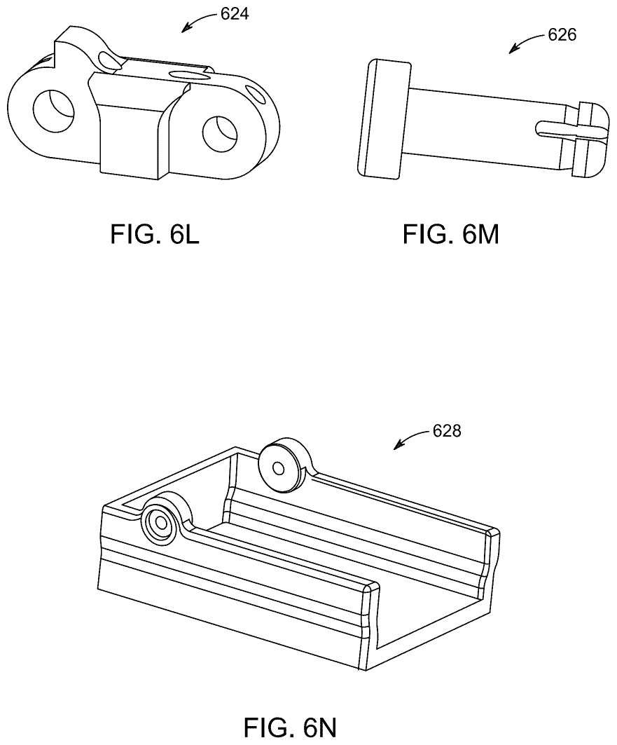

According to an aspect, in the case of designing an upper limb prototype, it involves the study of hand biomechanics, enlisting desired features in the prototype, material selection, and prototype development. This procedure resulted in the identification of 3D prototype structures and dimensions. The hand (right palm), hand connector, forearm, and wrist joint are the primary subsystem of the prototype and are independently developed. FIG. 6A depicts the right palm (represented by reference number “602”). FIG. 6B depicts the hand connector (represented by reference number “604”). FIG. 6C depicts the forearm, i.e., the forearm top (represented by reference number “606-1”) and the forearm bottom (represented by reference number “606-2”). FIG. 6D depicts the wrist joint (represented by reference number “608”).

All such parts were designed using SOLIDWORKS software. The prototype is produced from Polylactic Acid (PLA) plastic filaments. PLA is a thermoplastic biopolymer obtained from renewable sources, making it safe, ecofriendly, and suitable for medical devices. PLA is safe, cost-effective, easily printable, lightweight, ecofriendly, and strong. The production of PLA results in 68% fewer greenhouse gases emission and uses 65% less energy as well as being recyclable. In most cases, PLA is one of the most robust filaments utilized in 3D printing, with a 7250-psi tensile strength.

Since the experiment focuses on examining wrist movements, three parts for achieving such motions were addressed, namely, the wrist joint, wrist connector, and actuator base. FIG. 6E depicts the wrist connector (represented by reference number “610”). FIG. 6F depicts the actuator base (represented by reference number “612”). The actuator base is the base on which the two linear actuators are fixed and attached to a servo motor to rotate the base, the actuators, and the wrist in order to change the axis of movement by 90 degrees. The wrist joint is connected to the actuators allocated in the forearm. As a result, to exhibit a movement, the difference in the height of the two actuators will cause the wrist to tilt in the aimed direction. Further, FIG. 6G depicts the joint pin (represented by reference number “614”). FIG. 6H depicts the first finger (represented by reference number “616”). FIG. 6I depicts the proximal finger (represented by reference number “618”). FIG. 6J depicts the pin finger (represented by reference number “620”). FIG. 6K depicts the finger (represented by reference number “622”). FIG. 6L depicts the proximal thumb (represented by reference number “624”). FIG. 6M depicts the pin thumb (represented by reference number “626”). FIG. 6N depicts the circuit holder (represented by reference number “628”).

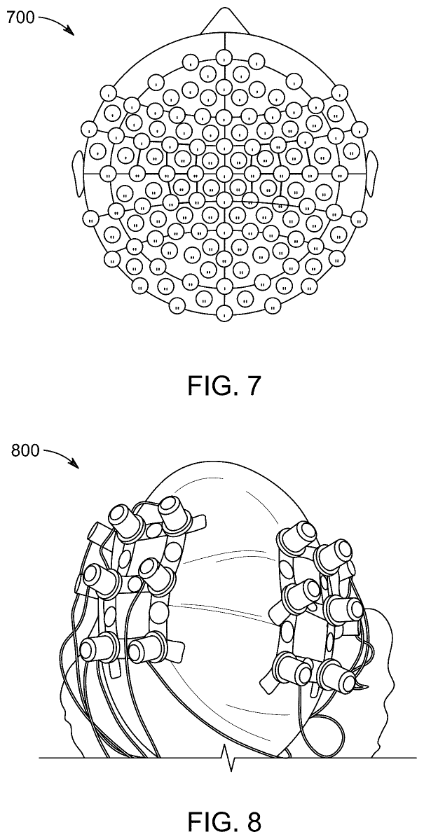

To acquire the hemodynamic response alternations, a Continuous Wave multi-channel fNIRS device (such as, fNIRS device 304), for example, NIRSport 8×8 (NIRx Medical Technologies, New York, NY, USA), was used, and the data was recorded at a sampling rate of 7.81 Hz. It was equipped with eight Light Emitting Diode (LED) sources (red) emitting near-infrared light with the power of 5 mW/wavelength at two wavelengths λ1|2=760,850 nm, and eight detectors resulting in 20 channels distributed symmetrically over the two hemispheres. FIG. 7 depicts an optodes arrangement 700, according to aspects of the present disclosure and FIG. 8 depicts another optodes arrangement 800, according to aspects of the present disclosure. The distance between the optodes was 3 cm. The data obtained is then converted to the corresponding hemoglobin concentrations by applying the Modified Beer-Lambert Law (MBLL).

Calibration is required prior to data collection to automatically calculate an appropriate amplification factor for each source-detector combination and to assess signal quality. The NIRStar program includes a signal quality check that marks the expected data quality for each channel using a simplified color-coded ‘traffic-light’ system. In this experiment, subjects looked for a signal quality that allows them to see the cardiac oscillations in the HbO signals clearly (for example, in the unfiltered preview display) and is suitable for the most demanding applications (for example, single-trial/single-subject evaluation). However, some data, such as the heart signal, may not be discernible in the raw display, but the noise level generally allows the extraction of neuro-activity using proper statistical evaluation.

Some characteristics of the fNIRS signal were addressed when creating the experimental paradigm. Since physiological artifacts dominate fNIRS data, each stimulation condition must be nearly duplicated multiple times to identify the functional response. Meanwhile, the hemodynamic response's temporal features impose constraints on the length of time between successive stimuli. Physiological artifacts which are related to the stimulus in time must also be considered. In examples, if the stimulation blocks are provided at regular intervals, a subject's breathing pattern may align with them, and this might lead to an increase in false-positive responses. These problems can be avoided by using an efficient experimental design to exclude anticipatory effects, such as pseudo-randomizing the sequence of conditions and the duration of the interstimulus duration.

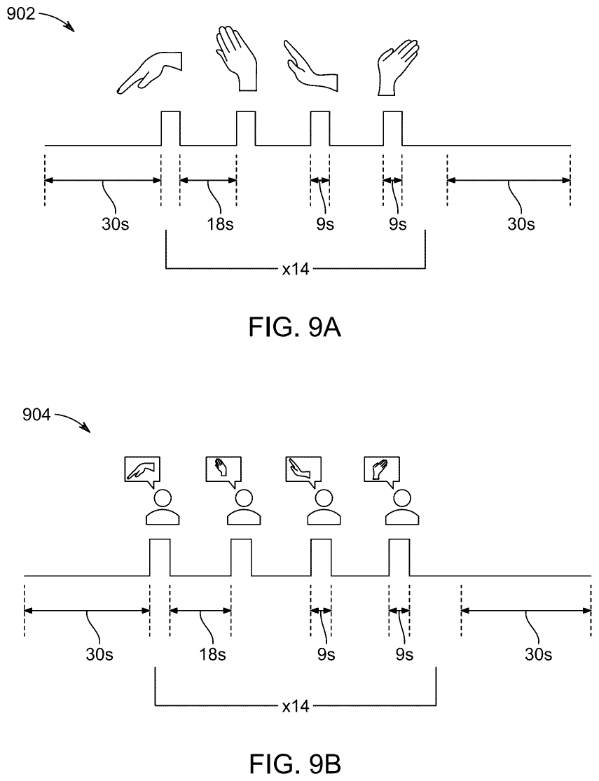

The experiment specifically focuses on the wrist movement, which are wrist extension, wrist flexion, ulnar deviation, and radial deviation. The experiment consisted of 4 trials, one movement per trial, each repeated 14 times. Throughout the experiment, the first 30 seconds were rest period to set up the baseline, followed by a trial of 9 seconds task period, which was followed in turn by another 18 seconds rest period, for a total trial duration of 25 minutes. The trials were shuffled and randomized using the Psychopy system. FIG. 9A and FIG. 9B illustrate the designed experimental paradigm of the experiment. FIG. 9A illustrates experimental paradigm 902 of real experiment and FIG. 9B illustrates experimental paradigm 904 of imagery experiment. Each subject repeated each task for four trials.

Further, each subject participated in two experiments, one for motor imagery and the other for motor execution which were taken on the same day. Each subject rested in a comfortable chair in a dimly lit room, watching a 14-inch computer at about 60 cm. The subject was instructed to rest for at least 5 minutes before doing the experiment to stabilize the pulse rate and blood pressure and to continue to be relaxed throughout the experiment to minimize excessive movement or thinking. During the experiment, each subject was instructed to imagine or perform a randomized movement of his or her right wrist depending on the suggested movement on the monitor. Prior to the actual experiment, all the subjects actively participated in a training session.

### Pre-Processing: Motion Artifacts and Filtering

The acquired fNIRS signals may include motion artifacts in the form of spikes or baseline shifts since fNIRS records not only brain activity during a task but also other signals (e.g., the noise of the measurement instrument, influences of breathing, and fluctuations in blood pressure). Such signals may include different types of noises, which are classified as instrumental noise, experimental error, and physiological noise. Instrumental noise refers to the noise of fNIRS signals in hardware, or that is produced by the environment, a low-pass filter (for instance, with a cut-off frequency of 35 Hz) may readily remove such a high frequency. Furthermore, instrumental noise may considerably be minimized by reducing the change of external light. Motion artifacts, such as head movements, cause optodes to move from their allotted places during the experiment. This can result in a spike-like noise due to a quick change in light intensity. Consequently, HbO and HbR are radically different from normal cases as the baseline changes. Signals typically also comprise physiological noises such as heartbeat (1-1.5 Hz), respiration (0.2-0.5 Hz), and low frequency content resulting from blood pressure fluctuations (Mayer waves; 0.1 Hz). Based on light absorption, signal conversion was performed using MBLL to obtain HbO and HbR data.

The output of the fNIRS system is a hemodynamic response that measures the blood delivery in the tissues in terms of HbO and HbR. The concentrations are obtained by detecting the reflected waves that take the banana shape at certain distances and path-length and converting the light intensity to optical density and then to concentration by applying the MBLL. This is illustrated in Equations (1), (2), and (3) provided below.

$$A = - {log}_{10} ( \frac{{I}_{out}}{{I}_{in}} ) \qquad ( 1 )$$

$$A = μ l = ε \cdot [ X ] \cdot d \cdot DPF + G \qquad ( 2 )$$

$$Δ A = ε \cdot [ X ] \cdot d \cdot DPF , \qquad ( 3 )$$

where A represents absorbance (also called optical density), Iₒᵤₜ represents light intensity of the detected wave, Iᵢₙ represents light intensity of the source, μ represents linear attenuation coefficient, l represents total path-length travelled by the photons, ε represents molar attenuation coefficient of that material, [X] represents chromophore concentration, d represents distance between source and detector, and DPF represents differential path-length factor.

As the total path traveled is affected by the distance between source and detector (d), which is fixed to be a constant value of 3 mm and differential path-length factor (DPF), the values will be different for the subject as their age differs. The older the subject, the larger the differential path-length factor. This difference is due to the aging process, such as the change in intracranial volume, cerebral blood flow and volume, myelination, cerebrospinal fluid layer thickness, or cortical thickness, bone mineral content, and cortical bone density. Since the HbO and HbR are required to be distinguishable, two wavelengths are used at each channel. In the NIRx system, the two wavelengths are 850 nm and 760 nm. One of the wavelengths is sensitive for HbO, and the other wavelength is sensitive for HbR. Also, the experiment was applied on subjects of age 22, so the DPF values were given as 5.0003 for 850 nm wavelength and 6.06258 for 760 nm wavelength. Therefore, the applied MBLL may be represented by Equations (4) and (5) provided below.

$$\frac{{A}_{λ 1}}{{l}_{λ 1}} = {ε}_{λ 1}^{HbO} \cdot [ HbO ] + {ε}_{λ 1}^{HbR} \cdot [ HbR ] \qquad ( 4 )$$

$$\frac{{A}_{λ 2}}{{l}_{λ 2}} = {ε}_{λ 2}^{HbO} \cdot [ HbO ] + {ε}_{λ 2}^{HbR} \cdot [ HbR ] \qquad ( 5 )$$

These Equations (4) and (5) are written in the form of matrices to solve them in terms of the concentrations, and the final form of concentration may be mathematically represented using Equations (6), (7), and (8) provided below.

$$[ \begin{matrix}[ {HbO}_{2} ] \\ [ Hb ]\end{matrix} ] = {[ \begin{matrix}{ε}_{λ 1}^{{HbO}_{2}} & {ε}_{λ 1}^{Hb} \\ {ε}_{λ 2}^{{HbO}_{2}} & {ε}_{λ 2}^{Hb}\end{matrix} ]}^{- 1} [ \begin{matrix}{A}_{λ 1} / {l}_{λ 1} \\ {A}_{λ 2} / {l}_{λ 2}\end{matrix} ] , \qquad ( 6 )$$

$$[ HbO ] = \frac{\frac{{A}_{λ 2}}{{λ}_{2}} {ε}_{λ 1}^{HbR} - \frac{{A}_{λ 1}}{{l}_{λ 1}} {ε}_{λ 2}^{HbR}}{{ε}_{λ 1}^{HbR} {ε}_{λ 2}^{HbO} - {ε}_{λ 2}^{HbR} {ε}_{λ 1}^{Hbo}} , \qquad ( 7 )$$

$$[ HbR ] = \frac{\frac{{A}_{λ 2}}{{l}_{λ 2}} {ε}_{λ 1}^{HbO} - \frac{{A}_{λ 1}}{{l}_{1}} {ε}_{λ 2}^{Hbo}}{{ε}_{λ 1}^{HbO} {ε}_{λ 2}^{HbR} - {ε}_{λ 2}^{HbO} {ε}_{λ 1}^{HbR}} , \qquad ( 8 )$$

HbO signals were found to be more suitable for categorization than HbR signals and total hemoglobin. As a result, HbO data was used to derive six different statistical features including Signal slope (SS), Signal Mean (SM), Signal Variance (SV), Signal Skewness (SW), Signal Kurtosis (SK), and Signal Minimum (SN). To achieve the classification accuracy, the two-features combination possibilities of SS, SM, SV, SW, SK, and SN were investigated.

All features were computed using built-in function in MATLAB program on all four tasks data points. ANN, SVM, and KNN classification algorithms were utilized to classify the two and three feature combinations. The KNN algorithm employs a weighted majority approach. It uses data from a training set to make predictions for fresh entries in the future. For each new record, the k-closest entries from the training data set are identified. The values of the targeted property of the nearby records are used to make a forecast for the new record. In addition, the input, hidden, and output layers of ANNs are made up of node layers. Each node, or artificial neuron, is linked to the next and has its own weight and sensitivity. Activation of a node occurs when its output reaches a specific level, and data is transferred to the next level of the network. Likewise, the SVM classifier classifies datasets by mapping them to a strong feature space, even when the data is not linearly separable. The data is transformed after establishing a separator between the categories so that the separator could be depicted as a hyperplane. The wrist flexion, wrist extension, ulnar deviation, and radial deviation classification accuracies among the twelve subjects were obtained using the MATLAB classification learner application. Four conditions were classified using quadrilateral classifications using 8-fold cross-validation to evaluate the classifications' performance.

To compute the features SS was determined by fitting a line on all the conditions data points and was computed by using the polyfit function in MATLAB. SM, SV, SW, and SK were calculated using Equations (9), (10), (11), and (12) provided below.

$$SM = \frac{1}{N} {\sum }_{i = 1}^{N} {X}_{i} . \qquad ( 9 )$$

$$SV ( X ) = \frac{\sum {( X - μ )}^{2}}{N} . \qquad ( 10$$

$$SW ( X ) = E [ {( \frac{X - μ}{σ} )}^{3} ] . \qquad ( 11 )$$

$$SK ( X ) = E [ {( \frac{X - μ}{σ} )}^{4} ] , \qquad ( 12 )$$

where N represents number of iterations, X represents HbO data, μ represents mean of X, ε represents expected value of X, and σ represents standard division of X.

### Channel Selection

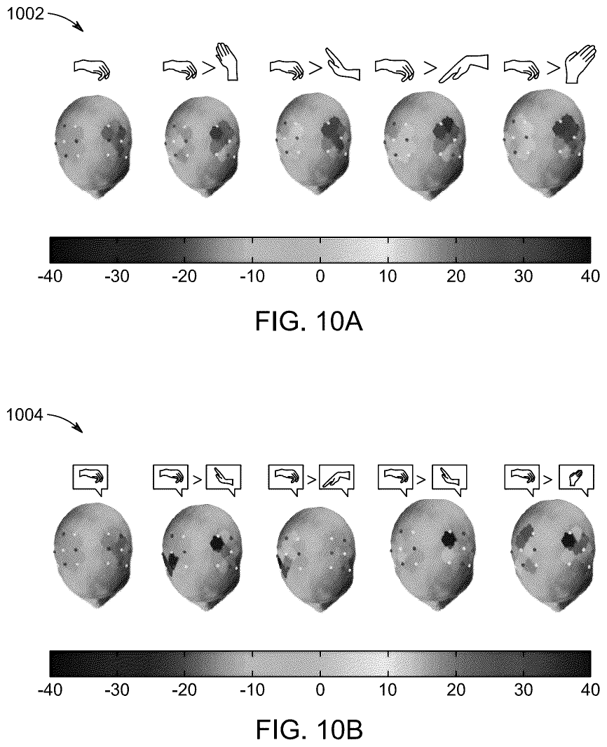

The mapping analysis of the acquired data from an individual healthy subject during wrist movement showed contralateral and bilateral activation of HbO in the motor cortex for both real and imagery experiments, respectively. During each task in the real and imagery experiments, HbO levels rise in the right and left hemispheres of the motor cortex. FIG. 10A illustrates an example 1002 of individual subject maps of HbO changes during real experiment of four conditions. FIG. 10A demonstrates the activation patterns associated with task execution events where the HbO activation patterns were significantly different between resting and doing the activities, according to the mapping produced from real experiments. In comparison to the right hemisphere, the left hemisphere showed strong activation, as shown in FIG. 10A.

Human brain studies indicate that hand tasks execution is associated with a greater hemoglobin concentration in the contralateral motor cortex. Accordingly, the left hemisphere of the brain is excited when the right hand is in motion. Therefore, the statistical characteristics of the real task experiment were extracted using channels 1 through 10.

FIG. 10B illustrates an example 1004 of individual subject maps of HbO changes during imagery experiment of four conditions. In all imagery tasks, the HbO was controlled in both hemispheres. The scale of HbO mapping in the real experiment is clearly larger than for the imagery experiment.

### Hemodynamic Response

The left and right hemispheres of the motor cortex are used to measure the hemodynamic response of twelve subjects. The data collected from all subjects reveals a clear HbO response generated by real wrist activities of wrist flexion, wrist extension, ulnar deviation, and radial deviation, as well as imagery wrist tasks.

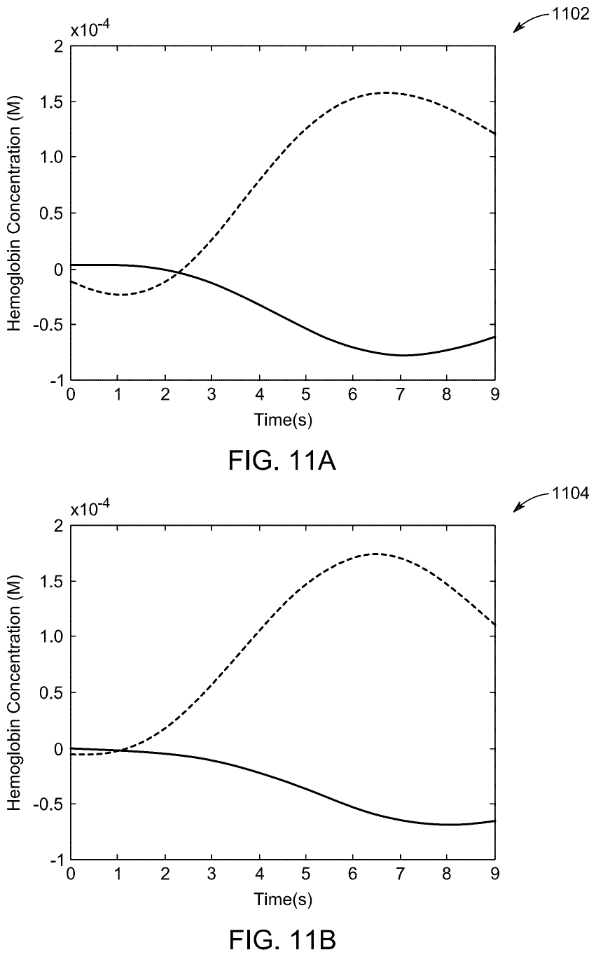

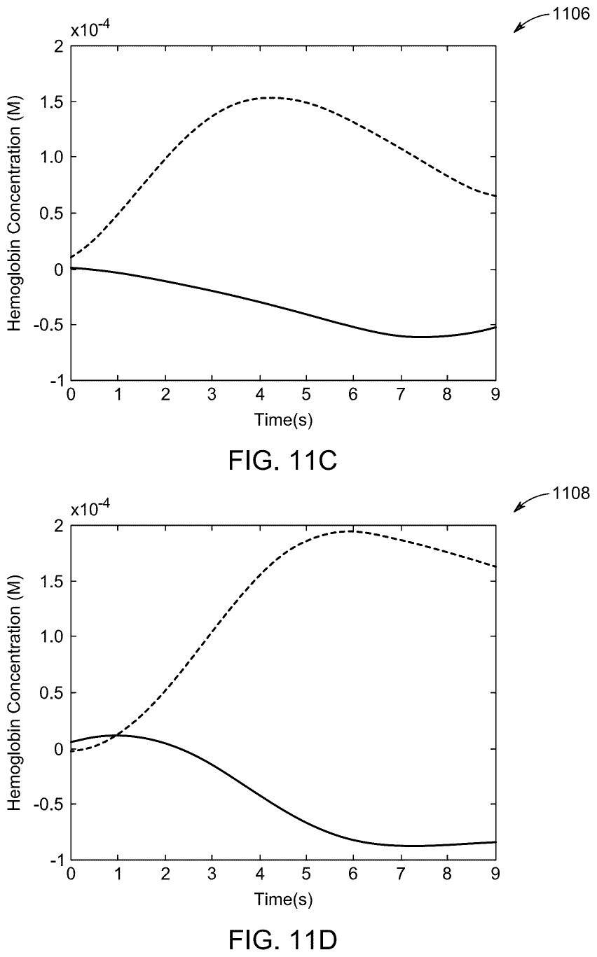

FIGS. 11A, 11B, 11C, and 11D show a typical hemodynamic response corresponding to real wrist tasks. In particular, FIGS. 11A, 11B, 11C, and 11D show HbO and HbR across left hemisphere channels of the averaged 12 subjects for all 14 trials for the four conditions of real experiments. FIG. 11A depicts a graphical plot 1102 illustrating a typical hemodynamic response corresponding to a wrist extension real task. FIG. 11B depicts a graphical plot 1104 illustrating a typical hemodynamic response corresponding to a wrist flexion real task. FIG. 11C depicts a graphical plot 1106 illustrating a typical hemodynamic response corresponding to a radian deviation real task. FIG. 11D depicts a graphical plot 1108 illustrating a typical hemodynamic response corresponding to an ulnar deviation real task.

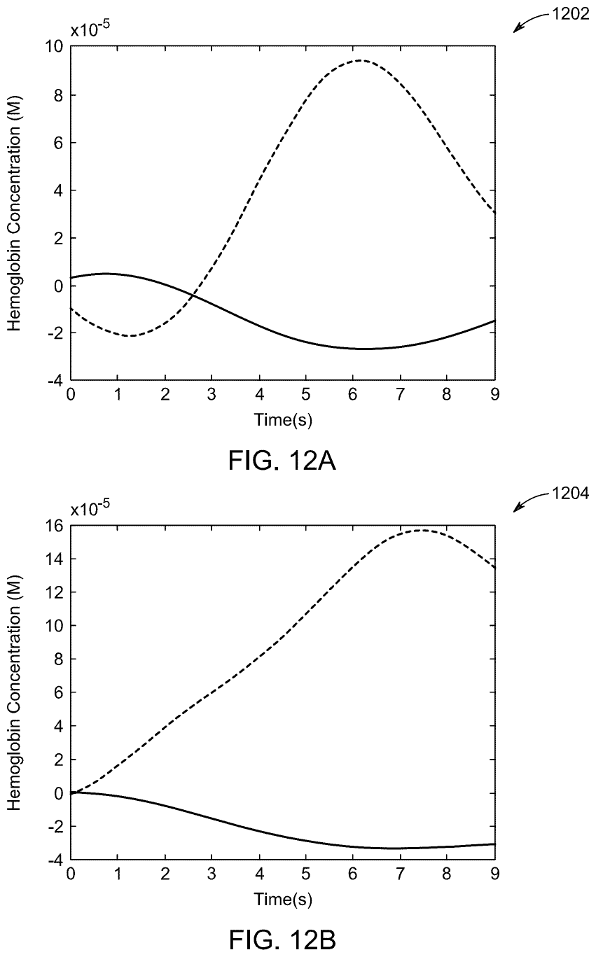

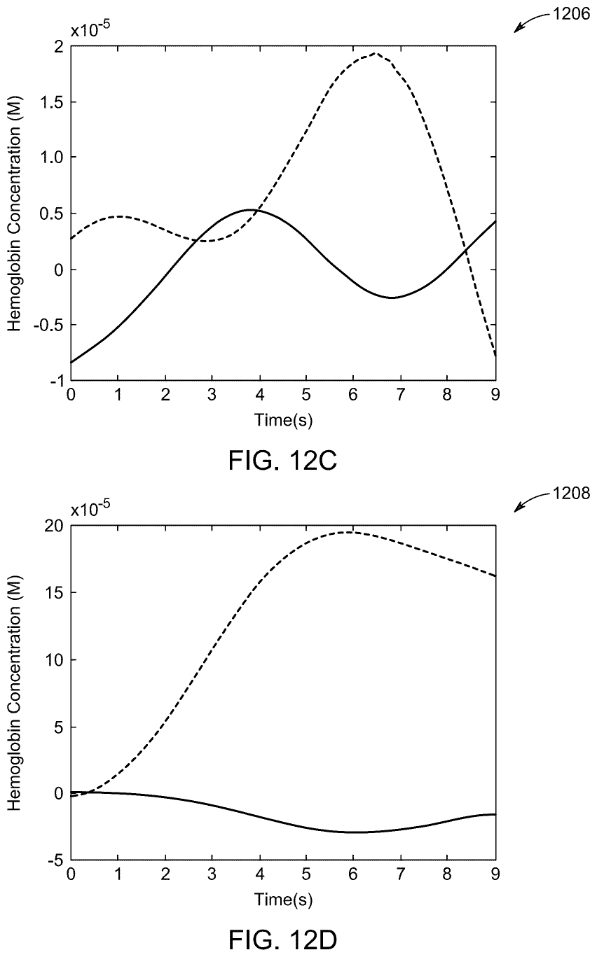

FIGS. 12A, 12B, 12C, and 12D show a typical hemodynamic response corresponding to imagery wrist tasks. FIGS. 12A, 12B, 12C, and 12D shows HbO and HbR for all channels of imagery tasks. FIG. 12A depicts a graphical plot 1202 illustrating a typical hemodynamic response corresponding to a wrist extension imagery task. FIG. 12B depicts a graphical plot 1204 illustrating a typical hemodynamic response corresponding to a wrist flexion imagery task. FIG. 12C depicts a graphical plot 1206 illustrating a typical hemodynamic response corresponding to a radian deviation imagery task. FIG. 12D depicts a graphical plot 1208 illustrating a typical hemodynamic response corresponding to an ulnar deviation imagery task. The highest value of HbO concentration in the imagery experiment is many times lower than in the real experiment.

As can be seen in FIGS. 11A, 11B, 11C, and 11D, ulnar deviation real task induced the highest concentration level of a value between (1.5×10⁻⁴-2×10⁻⁴) compared to other tasks and the imagery tasks that induced a concentration in the range of (1.5×10⁻⁵-2×10⁻⁵). The execution and imagery performed tasks showed variances in HbO time activation. As can be seen in FIGS. 11A, 11B, 11C, and 11D, the hemodynamic response in execution events experienced a great peak from 5 seconds to 7 seconds. However, imagery activation appeared around a time span of 6 seconds to 8 seconds. Since real movements generate consistent somatosensory and visual feedback in addition to muscle activation that is not present during imagery activities, differences between real and imagery tasks are expected and thus can be seen in the results.

### Classifications Accuracies

To achieve the greatest classification accuracy following a bandpass filtering of HbO signals, three classifiers were compared and 15 of two features extraction-combination. 8-fold cross-validation was used to create, train and validate the model. The accuracies of classifiers to differentiate flexion, extension, radial deviation, and ulnar deviation conditions during execution and imagery experiments are shown in tables 3 and 4, respectively. Accuracies were obtained from sitting the data of 12 subjects in a matrix as one dataset and sent as an input for each classifier.

TABLE 3
Real wrist tasks classification accuracy among 12 subjects

| | Accuracy (%) | | |
|---|---|---|---|
| Features | KNN (N = 1) | ANN | SVM |
| SS, SM | 70.8 | 77.1 | 70.8 |
| SS, SV | 72.9 | 68.8 | 60.4 |
| SS, SW | 79.8 | 72.9 | 72.9 |
| SS, SK | 77.1 | 75.0 | 75.0 |
| SS, SN | 70.8 | 70.8 | 56.2 |
| SM, SV | 72.9 | 75.0 | 68.8 |
| SM, SW | 70.8 | 77.1 | 58.3 |
| SM, SK | 75.0 | 68.1 | 66.7 |
| SM, SN | 75.0 | 75.0 | 62.5 |
| SV, SW | 72.9 | 77.1 | 70.8 |
| SV, SK | 72.9 | 75.0 | 54.0 |
| SV, SN | 79.2 | 79.2 | 56.2 |
| SW, SK | 75.0 | 81.2 | 68.8 |
| SW, SN | 72.9 | 75.0 | 56.2 |
| SK, SN | 70.8 | 72.9 | 52.1 |

TABLE 4
Imagery wrist tasks classification accuracy among 12 subjects

| | Accuracy (%) | | |
|---|---|---|---|
| Features | KNN (N = 1) | ANN | SVM |
| SS, SM | 58.3 | 62.5 | 41.7 |
| SS, SV | 54.2 | 58.3 | 35.4 |
| SS, SW | 60.4 | 64.4 | 50 |
| SS, SK | 58.3 | 60.4 | 58.3 |
| SS, SN | 62.5 | 64.4 | 54.2 |
| SM, SV | 60.4 | 64.6 | 43.8 |
| SM, SW | 56.2 | 64.6 | 50 |
| SM, SK | 64.6 | 66.7 | 60.4 |
| SM, SN | 66.7 | 62.5 | 56.2 |
| SV, SW | 60.4 | 58.3 | 43.8 |
| SV, SK | 62.5 | 68.8 | 47.9 |
| SV, SN | 64.6 | 66.7 | 45.8 |
| SW, SK | 58.3 | 60.4 | 43.8 |
| SW, SN | 58.3 | 58.3 | 52.1 |
| SK, SN | 62.5 | 68.8 | 50 |

From the data presented in tables 3 and 4, the presence of signal skewness in the two-feature combination was shown to produce the highest classification accuracies. Tables 3 and 4 show that, when compared to other modalities, ANN provides the best classification accuracies. Tables 5.1 and 5.2 summarizes the highest classification accuracies. SK/SW provided a high accuracy result in real and experiments, whereas SV/SK, SK/SN combination offered the best result in the imagery experiment. Accuracies of 81.2% and 68.8% are achieved along with ANN classifier for real and imagery experiments, respectively. One possible explanation for the lower accuracy in the imaging experiment compared to the real experiment is that the signal strength of the imagery tasks is lower than its equivalent in the real experiment. Furthermore, the participants may have completed the tasks at a different time in each trial during the imagery experiment, resulting in significant difficulties in identifying the onset of task signals, which is critical in not getting a similar extracted statistical feature across all trials of each task and thus reducing classification accuracies.

TABLE 5.1
Summary of the best classification accuracies

| | |
|---|---|
| Preference | Task |

Execution

| | | | |
|---|---|---|---|
| Features | SK, SW | SS, SW | SV, SN |
| Classifier | ANN | KNN | ANN, KNN |
| Accuracy (%) | 81.2 | 79.8 | 79.2 |

TABLE 5.2
Summary of the best classification accuracies

| | |
|---|---|
| Preference | Task |

Imagery

| | | | | |
|---|---|---|---|---|
| Features | SV, SK | SK, SN | SM, SK | SM, SN |
| | | | SV, SN | |
| Classifier | ANN | ANN | ANN, ANN | KNN |
| Accuracy (%) | 68.8 | 68.8 | 66.7 | 66.7 |

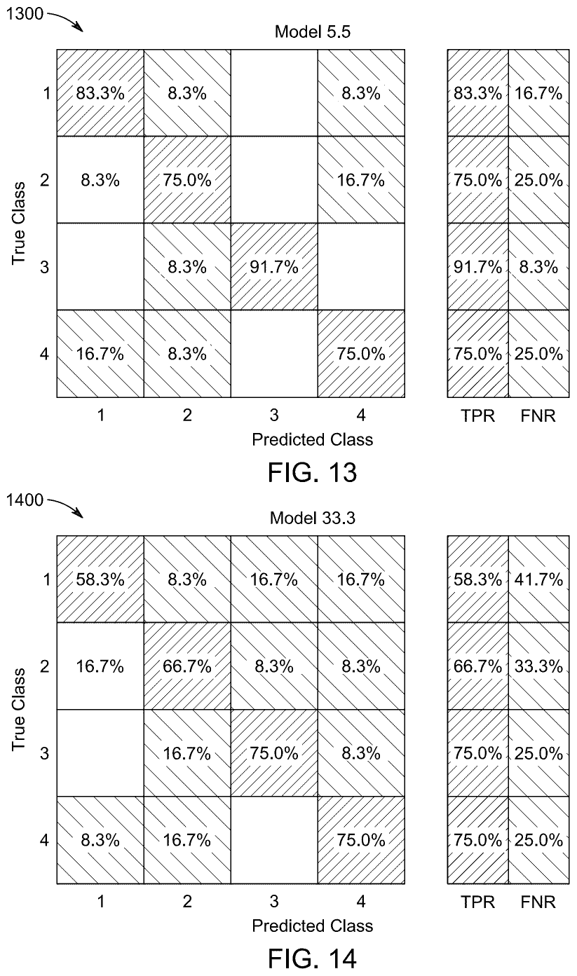

Multiple performance measures can be used to evaluate each classifier. The confusion matrix performance measurements, on the other hand, are the subject of the experiment. A confusion matrix is a table that visualizes two parameters, True Positive (TP) and False Negative (FN), to define and illustrate the projected results. The number of right and unsuccessful predictions are split down by class and presented with accuracy values. The actual class is defined by each row of the matrix, while the anticipated class is represented by the columns. The best accuracy scores based on the models provided in tables 5.1 and 5.2 were compared by plotting the confusion matrices corresponding to real and imagery experiments as shown in FIG. 13 and FIG. 14, respectively. FIG. 13 depicts real wrist tasks confusion matrix 1300, where classes labeled as 1, 2, 3, 4 stand for extension, flexion, radial deviation, and ulnar deviation classes, respectively. FIG. 14 depicts imagery wrist tasks confusion matrix 1400, where classes labeled as 1, 2, 3, 4 stand for extension, flexion, radial deviation, and ulnar deviation classes, respectively. It is evident that the radial deviation real task has the highest predicted class accuracy compared to other conditions, with an accuracy of 91.7%. The imagery confusion matrix established radial deviation and ulnar deviation as the best predicted classes with an accuracy of 75%.

### Cost Analysis

In tables 6 and 7, the cost is analyzed including rate, quantity, and total cost.

TABLE 6
Equipment Items costs analysis

| | | Rate | | Amount |
|---|---|---|---|---|
| No. | Description | (SAR) | Quantity | (SAR) |
| 01 | MG996R High torque Servo | 69 | 2 | 138 |
| | Motor 180° | | | |
| 02 | Arduino UNO R3 | 148.5 | 1 | 148.5 |
| 03 | L298N Motor driver module | 25.63 | 1 | 25.63 |
| 04 | 18650 Lithium Battery Shield | 34.5 | 1 | 34.5 |
| | For one battery | | | |
| 05 | 3.7 V 5000 mA Lithium Battery | 23 | 1 | 23 |
| 06 | BreadBoard | 11.5 | 1 | 11.5 |
| 07 | Wire Roll 100 m | 57.5 | 1 | 57.5 |
| 08 | Mini Electric Linear Actuator | 88.6 | 2 | 177.20 |
| | Stroke 5″-Force 4.5 lbs-12 V | | | |
| 09 | Uno R3 Case Enclosure | 37.7 | 1 | 37.7 |
| 10 | Lecxo 18650 battery 3600 mAh | 33.90 | 1 | 33.90 |
| | 3.7 v | | | |
| 11 | USB Printer Cable USB 2.0 Type | 29 | 1 | 29 |
| | A Male to B Male | | | |
| 12 | Jumper Wires 10 cm | 11.5 | 1 | 11.5 |

| | |
|---|---|
| Total | 727.93 SAR |

TABLE 7
Software items costs analysis

| Part | | Rate | | Amount |
|---|---|---|---|---|
| No. | Description | (SAR) | Quantity | (SAR) |
| SV | MATLAB and Simulink Student | 206.3 | 1 | 206.3 |
| | Suite | | | |
| NN | Deep Learning Toolbox | 6 | 1 | 22.51 |
| ME | MATLAB Coder | 6 | 1 | 22.51 |
| PO | Fixed-Point Designer | 6 | 1 | 22.51 |
| RL | Reinforcement Learning | 6 | 1 | 22.51 |
| | Toolbox | | | |
| Total | 296.34 SAR | | | |
| 3D | 650 SAR | | | |
| Printing | | | | |

### LIST OF STANDARDS

The standards considered during the experiment are given below.
A) IEC 80601-2-71:2015 Medical electrical equipment—Part 2-71: Requirements for the basic safety and essential performance of functional Near-Infrared Spectroscopy (fNIRS) equipment
B) ISO 16645:2016 Radiological protection—Medical electron accelerators—Requirements and recommendations for shielding design and evaluation
C) ISO/ASTM AWI 52933 Additive manufacturing—Environment, health, and safety—Consideration for the reduction of hazardous substances emitted during the operation of the non-industrial ME type 3D printer in workplaces, and corresponding test method

Based on the digital Computer-Aided Design (CAD) model, the structures are directly assembled into Standard Triangle Language file format for 3D printing. To be able to manufacture the design, a manufacturing company offering 3D printing created the physical object from the scaled three-dimensional digital model. The prototype parts were joined, and the electrical circuit components were placed in their functioning positions after acquiring the printed model.

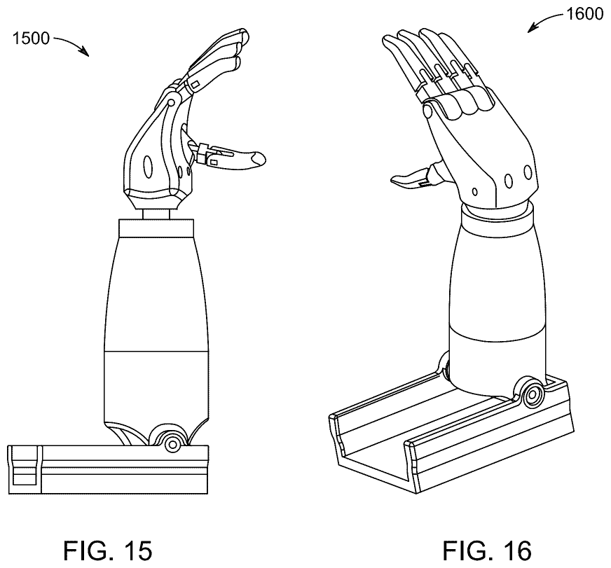

FIG. 15 shows a digital prototype 1500 (i.e., planned design) and FIG. 16 shows a real prototype 1600. The design can be wireless by using a Bluetooth module to send the commands to the Arduino microcontroller and get rid of any wire connection to achieve a fully portable design.

### Verification Test Description

To test and verify the classification result, a single trial data of each condition was obtained randomly from four subjects giving a total of 16 trials. The data was then processed based on predefined criteria (for example, motion artifacts). Besides, the built-in functions were implied to extract numerical data. Furthermore, the exported trained Model (ANN) structure from Classification Learner was used to make predictions using the new numerical data to identify each condition. The structure contains a classification object and a function for prediction “Test.predictFcn(x)”, where x is a row of 6 features arranged in columns of exactly the same order and format as the training data. The structure allows predictions for models that include Principal Component Analysis (PCA).

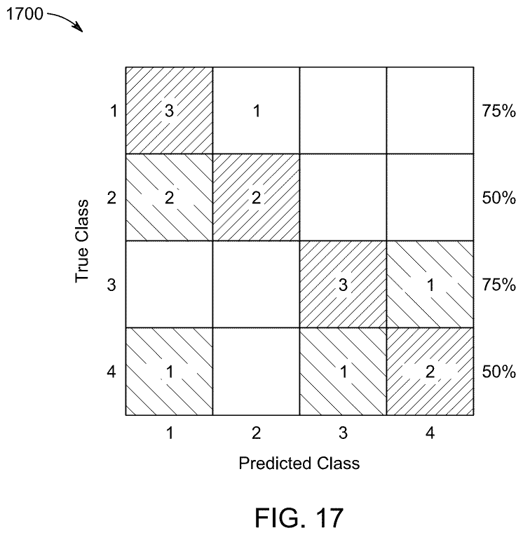

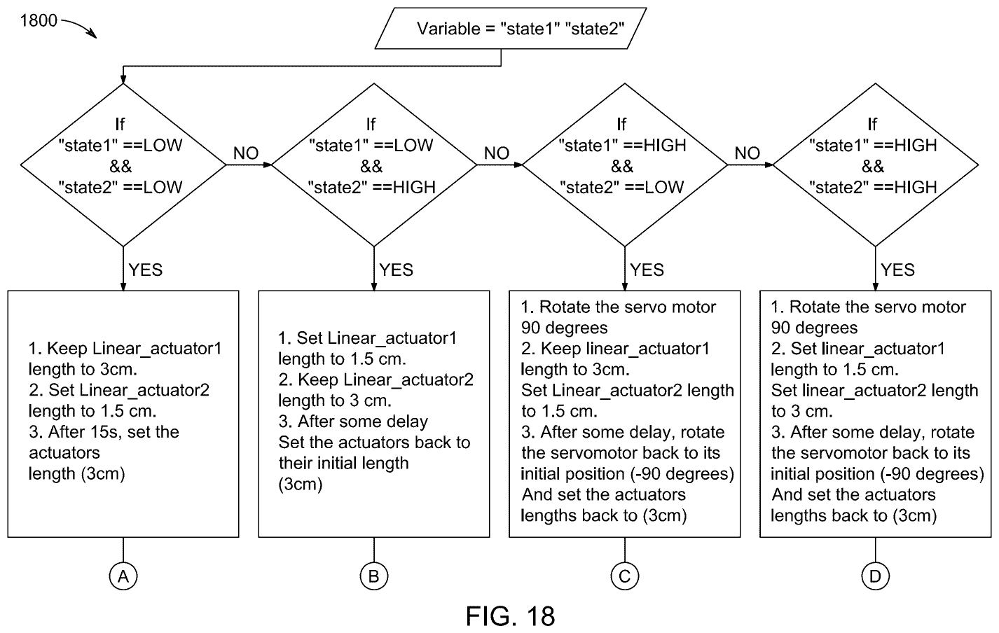

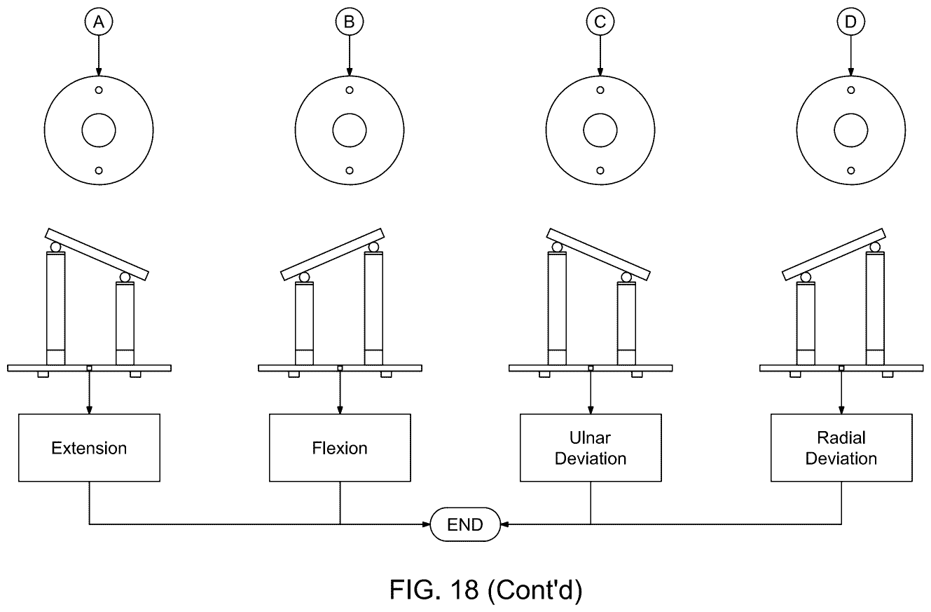

The evaluation of wrist flexion, wrist extension, radial, and ulnar deviation classification techniques can be obtained in terms of condition correctness by computing statistical measures, thus, the formation of a confusion matrix. These prediction results were identified per class, as shown in FIG. 17. FIG. 17 shows a confusion matrix 1700 of the predicted class, according to aspects of the present disclosure. The proper classifications are along the diagonal, whereas all of the other entries exhibit some misclassifications. As described in FIG. 17, the class 1 and class 3 achieved the highest accuracy of 75%, which will increase the probability for both classes to be correctly identified. The higher accuracy values prove a significant activation in the hemodynamic response during most trials for both classes. Accordingly, the ANN classification with two features combination technique consistently scores lower error. FIG. 18 describes example 1800 of wrist movement development, according to aspects of the present disclosure.

As described in FIG. 18, there are two variables, i.e., state 1 and state 2. In one scenario, state 1 and state 2 may be low. If this scenario, the length of a first linear actuator is kept as 3 cm and length of a second actuator is kept as 1.5 cm. After 15 seconds, the length of actuators is set back to their initial length (i.e., 3 cm). In another scenario, state 1 may be low and state 2 may be high. In this scenario, the length of the first linear actuator is kept as 1.5 cm and length of the second actuator is kept as 3 cm. After some delay, the actuators length is set back to their initial length (i.e., 3 cm). In yet another scenario, state 1 may be high and state 2 may be low. In this scenario, the servo motor is rotated at 90 degrees. The length of the first linear actuator is kept as 1.5 cm and length of the second actuator is kept as 3 cm. After some delay, the servo motor is rotated back to its initial position (−90 degrees) and the actuator length is set back to 3 cm. Further, in yet another scenario, state 1 may be high and state 2 may be high. In this scenario, the servo motor is rotated at 90 degrees. The length of the first linear actuator is kept as 3 cm and length of the second actuator is kept as 1.5 cm. After some delay, the servo motor is rotated back to its initial position (−90 degrees) and the actuator length is set back to 3 cm.

Further, feature extraction finds the most distinguishing properties in the HbO signals, making it easier for the designed machine learning to consume them. MATLAB built-in functions were used to accomplish the required six numerical features. When transitioning from raw data to constructing machine learning algorithms, this technique came in handy. Accordingly, the data of 12 subjects was set in a matrix as one dataset and sent as an input for each classifier.

The Classification Learner application was utilized to perform machine learning for testing, training models, and classifying the four conditions. After training KNN, ANN, and SVM models, their validation errors and performances were assessed to export the ANN algorithm, which is the best classification model. The exported function was applied to the matrix to get a structure field containing a prediction function. A sample input data was tested by calculating the six features followed by plugging in the prediction function to identify such input. The MATLAB code used to perform the above actions is provided below.

```
% Feature Extraction
subjects = 12;
condition1=load(‘Execution_Extention_Oxy.txt’);
condition2=load(‘Execution_Flection_Oxy.txt’);
condition3=load(‘Execution_RD_Oxy.txt’);
condition4=load(‘Execution_UD_Oxy.txt’);
% signal mean
for k=1:4
    Mean.(strcat(‘m’,num2str(k)))=
mean(eval(strcat(‘condition’,num2str(k))));
end
% signal Variance
for k=1:4
  Variance.(strcat(‘v’,num2str(k)))=
var(eval(strcat(‘condition’,num2str(k))));
end
% signal Kurtosis
for k=1:4
  Kurtosis.(strcat(‘k’,num2str(k)))=
kurtosis(eval(strcat(‘condition’,num2str(k))));
end
% signal Skewness
for k=1:4
  Skewness.(strcat(‘s',num2str(k)))=
skewness(eval(strcat(‘condition’,num2str(k))));
end
% signal Minimum
for k=1:4
  Minimum.(strcat(‘i’,num2str(k)))=
min(eval(strcat(‘condition’,num2str(k))));
end
% signal slope
for k=1:4
  par_slope = strcat(‘l’,num2str(k));
  Slope.(par_slope)= [ ];
  read1=eval(strcat(‘condition’,num2str(k)));
  for sub=1:subjects
      [a,b]=size(read1);
      x=linspace(0,9,a);
      Poly= polyfit(x.′,read1(:,sub),1);
      l1 = Poly(1);
      Slope.(par_slope)=[Slope.(par_slope);l1];
  End
end
% Generate Matrix
cond1=[Mean.m1.′,Variance.v1.′,Kurtosis.k1.′,Skewness.s1.′,Minimum.i
1.′,Slope.l1,ones(subjects,1)];
cond2=[Mean.m2.′,Variance.v2.′,Kurtosis.k2.′,Skewness.s2.′,Minimum.i
2.′,Slope.l2,2.*ones(subjects,1)];
cond3=[Mean.m3.′,Variance.v3.′,Kurtosis.k3.′,Skewness.s3.′,Minimum.i
3.′,Slope.l3,3.*ones(subjects,1)];
cond4=[Mean.m4.′,Variance.v4.′,Kurtosis.k4.′,Skewness.s4.′,Minimum.i
4.′,Slope.l4,4.*ones(subjects,1)];
Features=[ ];
for f=1:subjects
      for l=1:4
        read=eval(strcat(‘cond’,num2str(l)));
        Features=[Features;read(f,:)];
        end
end
  % ANN classifer function
        test=trainClassifier(Features);
% Request user input to load a condition
      promt=‘enter the desired condition (write the full file extension
      inside ″)’;
      cond=input(promt);
      cond=load(cond);
      [r,c]=size(cond);
      if ~(c == 1)
        disp(‘error: the file contains more than one cloumn’)
        else
          % Extract the condition Features
          p=linspace(0,9,r);
          fit= polyfit(p.′,cond,1);
          condition_features=[mean(cond),var(cond),kurtosis(cond),skew
ness(cond),min(cond),fit(1)];
          % predict the input class
          Prediction = test.predictFcn(condition_features) % predict the input class
          end
      % link the condition to arduino
      ar = arduino(‘COM3’, ‘Uno’);
      writeDigitalPin(ar, ‘D11’, 0);
      writeDigitalPin(ar, ‘D12’, 1);
      if prediction==1
      writeDigitalPin(ar, ‘D11’, 0);
      writeDigitalPin(ar, ‘D12’, 0);
      elseif prediction==2
      writeDigitalPin(ar, ‘D11’, 1);
      writeDigitalPin(ar, ‘D12’, 0);
      elseif prediction==3
      writeDigitalPin(ar, ‘D11’, 0);
      writeDigitalPin(ar, ‘D12’, 1);
      elseif prediction==4
      writeDigitalPin(ar, ‘D11’, 1);
      writeDigitalPin(ar, ‘D12’, 1);
      else disp(‘error’ ) end
```

The ANN classifier function used to perform the above actions is provided below.

```
function [trainedClassifier, validationAccuracy] =
trainClassifier (trainingData)
% [trainedClassifier, validationAccuracy] =
trainClassifier(trainingData)
% Returns a trained classifier and its accuracy. This code recreates
the
% classification model trained in Classification Learner app. Use
the
% generated code to automate training the same model with new data,
or to
% learn how to programmatically train models.
%
% Input:
%  trainingData: A matrix with the same number of columns and
data type
% as the matrix imported into the app.
%
% Output:
% trainedClassifier: A struct containing the trained
classifier. The
% struct contains various fields with information about the
trained
% classifier.
%
% trainedClassifier.predictFcn: A function to make predictions
on new
% data.
%
% validationAccuracy: A double containing the accuracy as a
% percentage. In the app, the Models pane displays this
overall
% accuracy score for each model.
%
% Use the code to train the model with new data. To retrain your
% classifier, call the function from the command line with your
original
% data or new data as the input argument trainingData.
%
% For example, to retrain a classifier trained with the original
data set
% T, enter:
% [trainedClassifier, validationAccuracy] = trainClassifier(T)
%
% To make predictions with the returned ‘trainedClassifier’ on new data
T2,
% use
% yfit = trainedClassifier.predictFcn(T2)
%
% T2 must be a matrix containing only the predictor columns used for
% training. For details, enter:
% trainedClassifier.HowToPredict
% Auto-generated by MATLAB on 27-Apr-2022 00:48:01
% Extract predictors and response
% This code processes the data into the right shape for training the
% model.
% Convert input to table
inputTable = array2table(trainingData, ‘VariableNames’, {‘column_1’,
‘column_2’, ‘column_3’, ‘column_4’, ‘column_5’, ‘column_6’,
‘column_7’});
predictorNames = {‘column_1’, ‘column_2’, ‘column_3’, ‘column_4’,
‘column_5’, ‘column_6’};
predictors = inputTable(:, predictorNames);
response = inputTable.column_7;
isCategoricalPredictor = [false, false, false, false, false, false];
% Data transformation: Select subset of the features
% This code selects the same subset of features as were used in the
app.
includedPredictorNames = predictors.Properties.VariableNames([false
false true true true false]);
predictors = predictors(:,includedPredictorNames);
isCategoricalPredictor = isCategoricalPredictor([false false true
true true false]);
% Train a classifier
% This code specifies all the classifier options and trains the
classifier.
classificationNeuralNetwork = fitcnet(...
  predictors, ...
  response, ...
  ‘LayerSizes’, [10 10 10], ...
  ‘Activations’, ‘relu’, ...
  ‘Lambda’, 0, ...
  ‘IterationLimit’, 1000, ...
  ‘Standardize’, true, ...
  ‘ClassNames’, [1; 2; 3; 4]);
% Create the result struct with predict function
predictorExtractionFcn = @(x) array2table(x, ‘VariableNames’,
predictorNames);
featureSelectionFcn = @(x) x(:,includedPredictorNames);
neuralNetworkPredictFcn = @(x) predict(classificationNeuralNetwork,
x);
trainedClassifier.predictFcn = @(x)
neuralNetworkPredictFcn(featureSelectionFcn(predictorExtractionFcn(x
)));
% Add additional fields to the result struct
trainedClassifier.ClassificationNeuralNetwork =
classificationNeuralNetwork;
trainedClassifier.About = ‘This struct is a trained model exported
from Classification Learner R2022a.’;
trainedClassifier.HowToPredict = sprintf(‘To make predictions on a
new predictor column matrix, X, use: \n yfit = c.predictFcn(X)
\nreplacing “c” with the name of the variable that is this struct,
e.g., “trainedModel”. \n \nX must contain exactly 6 columns because
this model was trained using 6 predictors. \nX must contain only
predictor columns in exactly the same order and format as your
training \ndata. Do not include the response column or any columns
you did not import into the app. \n \nFor more information, see <a
href=“matlab:helpview(fullfile(docroot, “stats”, “stats.map”),
“appclassification_exportmodeltoworkspace”)”>How to predict using
an exported model</a>.’);
% Extract predictors and response
% This code processes the data into the right shape for training the
% model.
% Convert input to table
inputTable = array2table(trainingData, ‘VariableNames’, {‘column_1’,
‘column_2’, ‘column_3’, ‘column_4’, ‘column_5’, ‘column_6’,
‘column_7’});
predictorNames = {‘column_1’, ‘column_2’, ‘column_3’, ‘column_4’,
‘column_5’, ‘column_6’};
predictors = inputTable(:, predictorNames);
response = inputTable.column_7;
isCategoricalPredictor = [false, false, false, false, false, false];
% Perform cross-validation
partitionedModel =
crossval(trainedClassifier.ClassificationNeuralNetwork, ‘KFold’, 8);
% Compute validation predictions
[validationPredictions, validationScores] =
kfoldPredict(partitionedModel);
% Compute validation accuracy
validationAccuracy = 1 - kfoldLoss(partitionedModel, ‘LossFun’,
‘ClassifError’);
```

The output of the sampled input data from MATLAB is plugged to control two shaft positions depending on the condition number. The Arduino code is given below.

| 1 | # include “setup.h” |
|---|---|
| 2 | # include <Servo.h> |
| 3 | |
| 4 | enum motor_position{CLOSE ,HALF ,OPEN}position; |
| 5 | Servo servo; // create servo object to control servo |
| 6 | |
| 7 | int pin11= 11; // pushbutton connected to digital pin 11 |
| 8 | int pin12= 12; // pushbutton connected to digital pin 12 |
| 9 | int state1= 0; |
| 10 | int state2= 0; |
| 11 | |
| 12 | |
| 13 | |
| 14 | |
| 15 | void setup( ) { |
| 16 | |
| 17 | Serial.begin(9600); |
| 18 | Serial.println(“MOTOR CONTROL INITIATED ”); |
| 19 | |
| 20 | servo.attach(3);// attaches the servo on pin 3 to the |
| 21 | servo object |
| 22 | pinMode (pin11, INPUT); // sets the digital pin 11 as |
| 23 | input |
| 24 | pinMode(pin12,INPUT); // sets the digital pin 12 as |
| 25 | input |
| 26 | pinMode (enA, OUTPUT); |
| 27 | pinMode(A1,OUTPUT); |
| 28 | pinMode(A2,OUTPUT); |
| 29 | pinMode(enB,OUTPUT); |
| 30 | pinMode(B1,OUTPUT); |
| 31 | pinMode(B2,OUTPUT); |
| 32 | |
| 33 | // Disable all motors |
| 34 | digitalWrite(A1, LOW); |
| 35 | digitalWrite(A2, LOW); |
| 36 | digitalWrite(B1, LOW); |
| 37 | digitalWrite(B2, LOW); |
| 38 | |
| 39 | } |
| 40 | |
| 41 | void loop( ) |
| 42 | { |
| 43 | |
| 44 | state1=digitalRead(pin11); |
| 45 | state2=digitalRead(pin12); |
| 46 | |
| 47 | //Extension |
| 48 | if ( state1 == LOW && state2 == LOW) |
| 49 | { |
| 50 | close_actuators( ); |
| 51 | } |
| 52 | |
| 53 | |
| 54 | //Flexion |
| 55 | else if (state1== LOW && state2 == HIGH ) |
| 56 | { |
| 57 | open_actuators( ); |
| 58 | } |
| 59 | |
| 60 | //Ulnar Deviation |
| 61 | else if (state1== HIGH && state2 == LOW) |
| 62 | { |
| 63 | for(pos = 0; pos <= 90; pos += 1)// sweeps |
| 64 | from 0 degrees to 90 degrees |
| 65 | { |
| 66 | servo.write(pos); // tell servo to go |
| 67 | to position in variable ‘pos’ |
| 68 | delay(15); // waits 15ms for the servo |
| 69 | to reach the position |
| 70 | } |
| 71 | close_actuators( ); |
| 72 | for(pos = 90; pos>=0; pos−=1)// sweeps from |
| 73 | 90 degrees to 0 degrees |
| 74 | { |
| 75 | servo.write(pos); // tell servo to go to |
| 76 | position in variable ‘pos’ |
| 77 | delay(15); // waits 15ms for the servo |
| 78 | to reach the position |
| 79 | } |
| 80 | |
| 81 | } |
| 82 | |
| 83 | //Radial Deviation |
| 84 | else if (state1== HIGH && state2 == HIGH) |
| 85 | { |
| 86 | for(pos = 0; pos <= 90; pos += 1)// sweeps |
| 87 | from 0 degrees to 90 degrees |
| 88 | { |
| 89 | servo.write(pos); // tell servo to go |
| 90 | to position in variable ‘pos’ |

91

delay(15); // waits 15ms for the servo

to reach the position

```
          } open_actuators( );
        //cond=“”;
      for(pos = 90; pos>=0; pos−=1)// sweeps from
```

90 degrees to 0 degrees

{ servo.write(pos); // tell servo to go to

position in variable ‘pos’

delay(15); // waits 15ms for the servo

to reach the position

```
          }
      }
```

}

The software tools used to stimulate, acquire, and process the fNIRS signals include NIRStar15-2, nirsLAB_v201904, and MATLAB R2017b softwares.

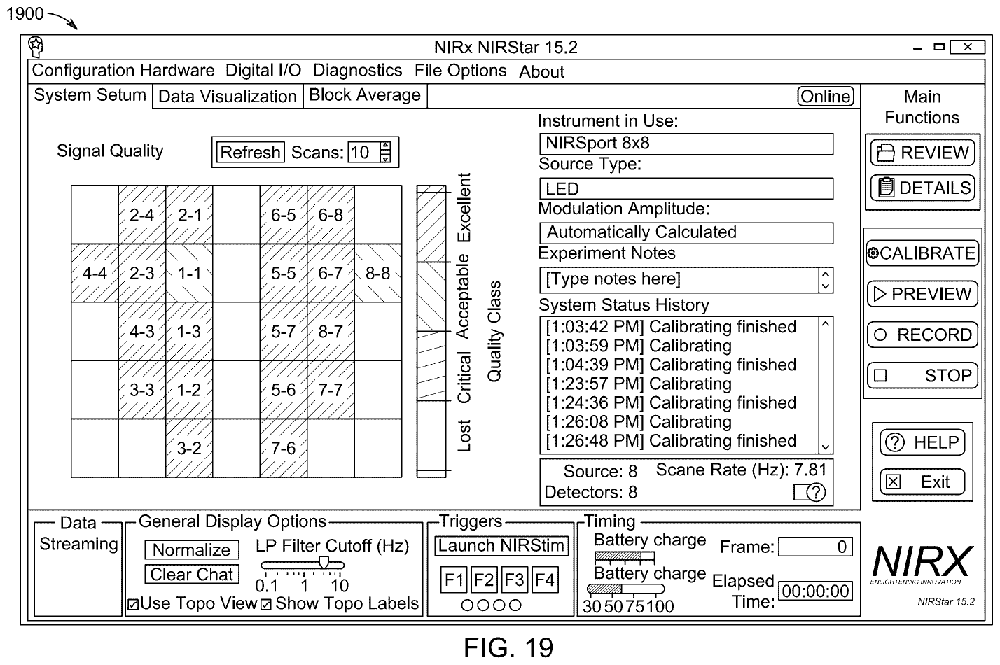

FIG. 19 depicts NIRStar15-2 interface 1900, according to aspects of the present disclosure. NIRStar15-2 is a controlling platform used to acquire and read raw fNIRS signals. NIRStar15-2 permits a high adaptability to various paradigms that can be investigated by other programs. NIRStar15-2 software obtains detailed information related to the experiment such as the hardware configuration, examination of quality metrics, channel selection, and test optodes saturation level.

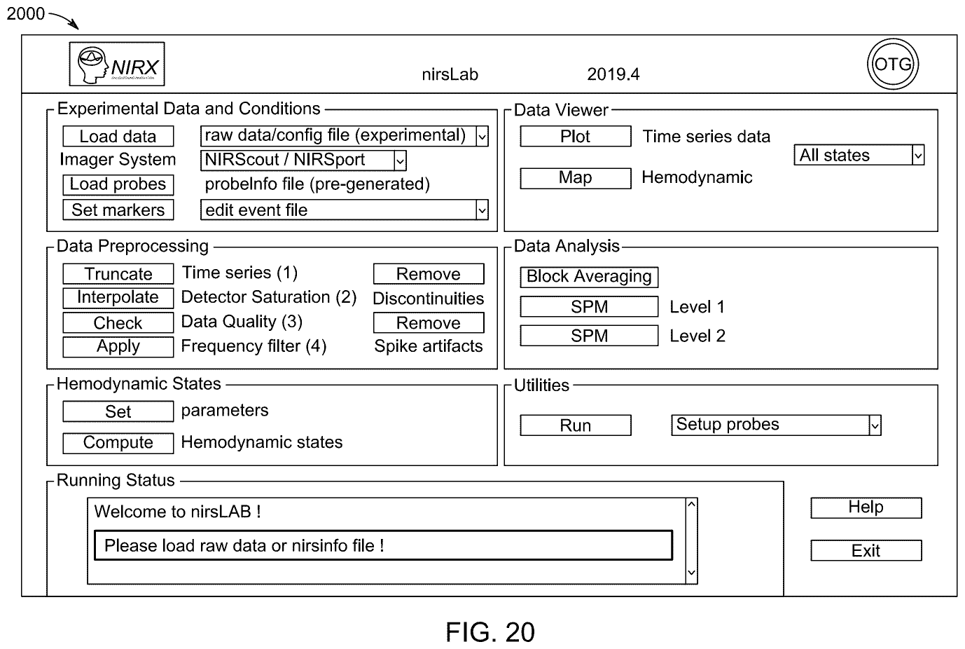

FIG. 20 depicts nirsLAB software interface 2000, according to aspects of the present disclosure. NirsLAB is a software that offers a rich statistical parametric mapping. This program was used to process the raw fNIRS signals into HbO and HbR, and map them on the brain surface.

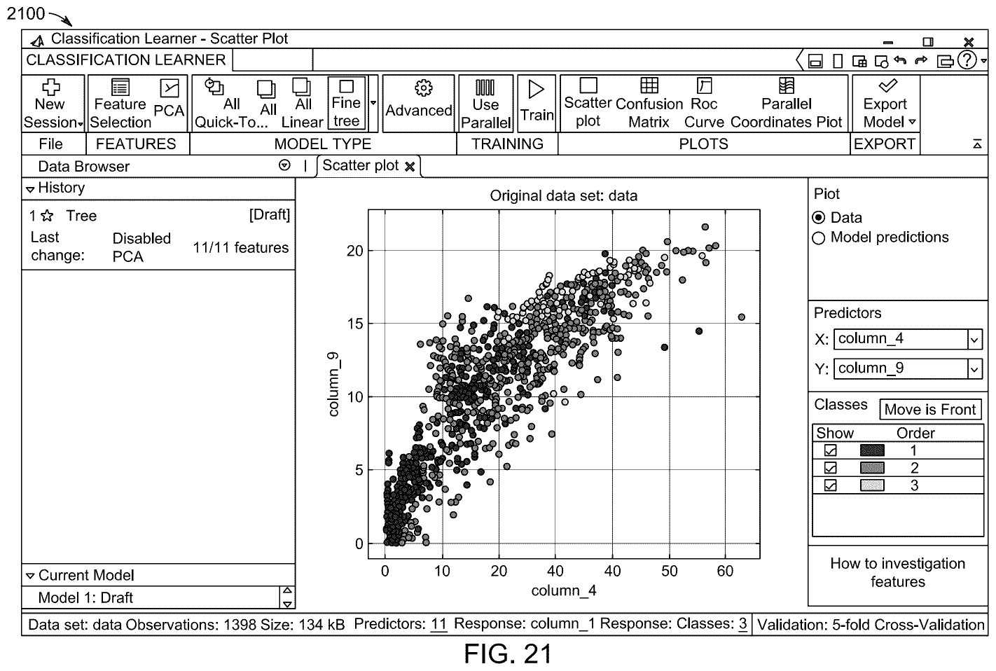

FIG. 21 depicts classification learner application interface 2100, according to aspects of the present disclosure. A special MATLAB built-in functions was used to extract some specific features of the HbO data. Those features were employed to classify the wrist conditions using the classification learner application.

According to aspect of the present disclosure, since the prototype is produced from PLA, a prosthetic hand using fNIRS will improve the quality of life of disabled people, increase patient independence and increase Patient engagement in the community.

The experiment demonstrates the feasibility of including such activities into the fNIRS-BCI system, so that amputees' patients' quality of life improves. fNIRS neuroimaging data was used to differentiate four activities of wrist movement and rest time. Further, two features-combination tests indicated that signal skewness and signal kurtosis represent one of the best two-combined features for producing high accuracy results of 81.2% employing ANN algorithm for real experiments. In imagery trials, however, signal kurtosis combined with either signal variance or signal minimum yielded the maximum accuracy of 68.8% utilizing the ANN algorithm.

Remarkably, the subjects showed clear induced HbO responses for both execution and imagery wrist activities. In a preliminary hand prototype, the system provides a degree of motion for a wrist's four main tasks; a processed command controls that. Indeed, the developed prototype proved to be viable enough for further development and clinical testing with patients in need of rehabilitation.

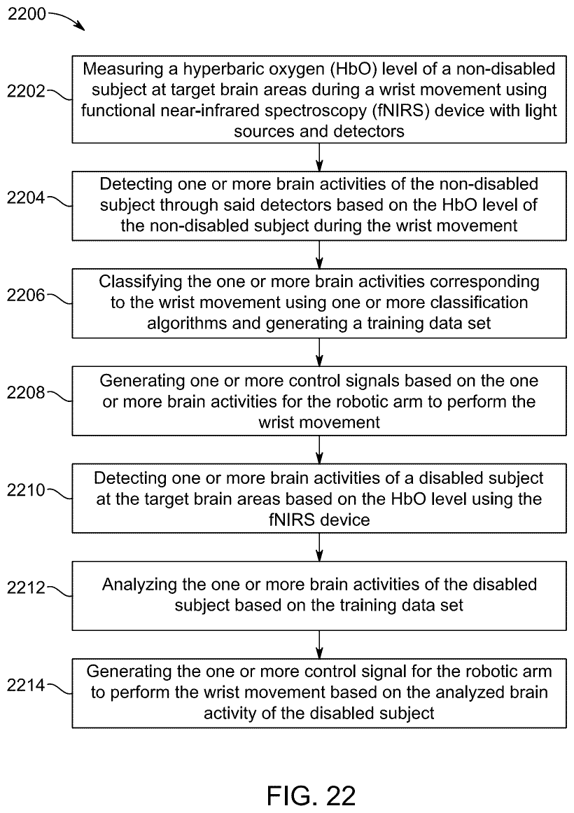

FIG. 22 illustrates a method 2200 for controlling a robotic arm 302 based on brain activities, according to aspects of the present disclosure.

At step 2202, the method 2200 includes measuring a HbO level of a non-disabled subject at target brain areas during a wrist movement using fNIRS device 304 with light sources 306 and detectors 306. The fNIRS device 304 includes eight light sources and eight detectors. In examples, the light sources 306 emit light at a peak emission of 760±10 nm and at a peak emission of 850±10 nm. Further, the wrist movement includes at least one selected from the group consisting of wrist extension, wrist flexion, ulnar deviation, and radial deviation of the non-disabled subject.

At step 2206, the method 2200 includes detecting one or more brain activities of the non-disabled subject through said detectors 308 based on the HbO level of the non-disabled subject during the wrist movement.

At step 2208, the method 2200 includes classifying the one or more brain activities corresponding to the wrist movement using one or more classification algorithms and generating a training data set. The one or more classification algorithms includes Artificial Neural Networks (ANN), K-Nearest Neighbor (KNN) or Support Vector Machine (SVM).

At step 2210, the method 2200 includes generating one or more control signals based on the one or more brain activities for the robotic arm 302 to perform the wrist movement.

At step 2212, the method 2200 includes detecting one or more brain activities of a disabled subject at the target brain areas based on the HbO level using the fNIRS device 304.

At step 2214, the method 2200 includes analyzing the one or more brain activities of the disabled subject based on the training data set.

At step 2214, the method 2200 includes generating the one or more control signal for the robotic arm 302 to perform the wrist movement based on the analyzed brain activity of the disabled subject.

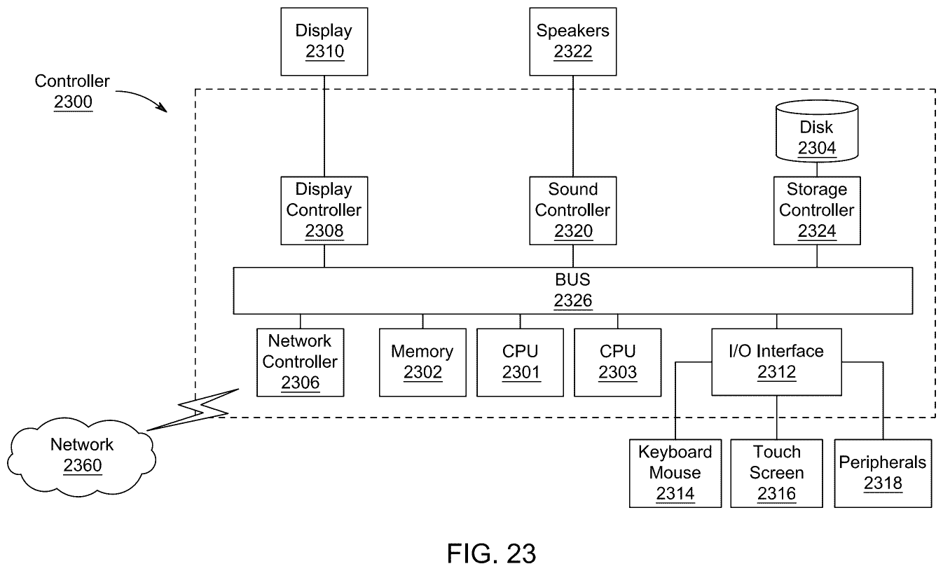

Next, further details of the hardware description of the computing environment according to exemplary embodiments is described with reference to FIG. 23. FIG. 23 is an illustration of a non-limiting example of details of computing hardware used in the computing system, according to exemplary aspects of the present disclosure. In FIG. 23, a controller 2300 is described which is a computing device (for example, the system 300) and includes a CPU 2301 which performs the processes described above/below. The process data and instructions may be stored in memory 2302. These processes and instructions may also be stored on a storage medium disk 2304 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 2301, 2303 and an operating system such as Microsoft Windows 7, UNIX, Solaris, LINUX, Apple MAC-OS, and other systems known to those skilled in the art.

The hardware elements in order to achieve the computing device may be realized by various circuitry elements, known to those skilled in the art. For example, CPU 2301 or CPU 2303 may be a Xenon or Core processor from Intel of America or an Opteron processor from AMD of America, or may be other processor types that would be recognized by one of ordinary skill in the art. Alternatively, the CPU 2301, 2303 may be implemented on an FPGA, ASIC, PLD or using discrete logic circuits, as one of ordinary skill in the art would recognize. Further, CPU 2301, 2303 may be implemented as multiple processors cooperatively working in parallel to perform the instructions of the inventive processes described above.

The computing device in FIG. 23 also includes a network controller 2306, such as an Intel Ethernet PRO network interface card from Intel Corporation of America, for interfacing with network 2360. As can be appreciated, the network 2360 can be a public network, such as the Internet, or a private network such as an LAN or WAN network, or any combination thereof and can also include PSTN or ISDN sub-networks. The network 2360 can also be wired, such as an Ethernet network, or can be wireless such as a cellular network including EDGE, 3G and 4G wireless cellular systems. The wireless network can also be WiFi, Bluetooth, or any other wireless form of communication that is known.

The computing device further includes a display controller 2308, such as a NVIDIA GeForce GTX or Quadro graphics adaptor from NVIDIA Corporation of America for interfacing with display 2310, such as a Hewlett Packard HPL2445w LCD monitor. A general purpose I/O interface 2312 interfaces with a keyboard and/or mouse 2314 as well as a touch screen panel 2316 on or separate from display 2310. General purpose I/O interface also connects to a variety of peripherals 2318 including printers and scanners, such as an OfficeJet or DeskJet from Hewlett Packard.

A sound controller 2320 is also provided in the computing device such as Sound Blaster X-Fi Titanium from Creative, to interface with speakers/microphone 2322 thereby providing sounds and/or music.

The general-purpose storage controller 2324 connects the storage medium disk 2304 with communication bus 2326, which may be an ISA, EISA, VESA, PCI, or similar, for interconnecting all of the components of the computing device. A description of the general features and functionality of the display 2310, keyboard and/or mouse 2314, as well as the display controller 2308, storage controller 2324, network controller 2306, sound controller 2320, and general purpose I/O interface 2312 is omitted herein for brevity as these features are known.

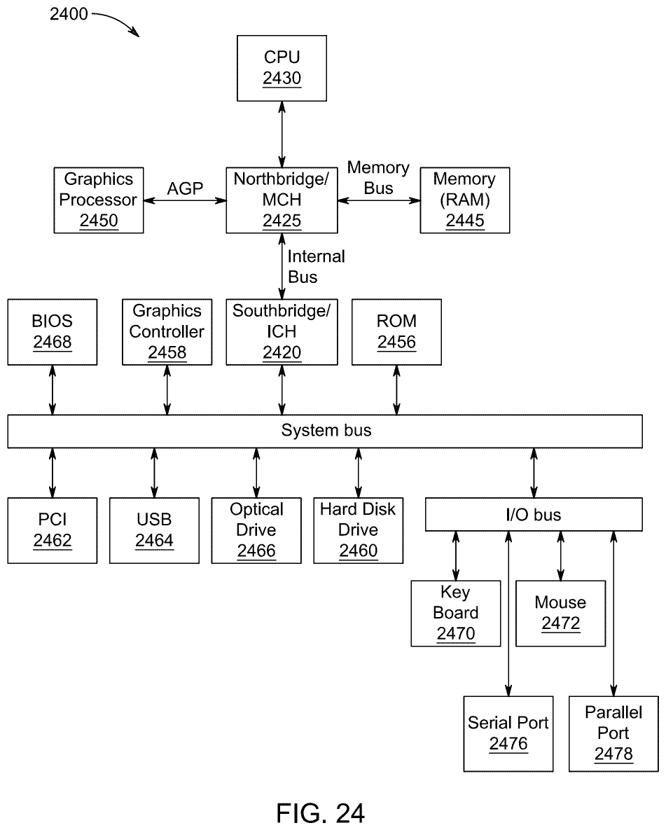

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 24.

FIG. 24 shows a schematic diagram of a data processing system 2400 used within the computing system, according to exemplary aspects of the present disclosure. The data processing system 2400 is an example of a computer in which code or instructions implementing the processes of the illustrative aspects of the present disclosure may be located.

In FIG. 24, data processing system 2400 employs a hub architecture including a north bridge and memory controller hub (NB/MCH) 2425 and a south bridge and input/output (I/O) controller hub (SB/ICH) 2420. The central processing unit (CPU) 2430 is connected to NB/MCH 2425. The NB/MCH 2425 also connects to the memory 2445 via a memory bus, and connects to the graphics processor 2450 via an accelerated graphics port (AGP). The NB/MCH 2425 also connects to the SB/ICH 2420 via an internal bus (e.g., a unified media interface or a direct media interface). The CPU Processing unit 2430 may contain one or more processors and even may be implemented using one or more heterogeneous processor systems.

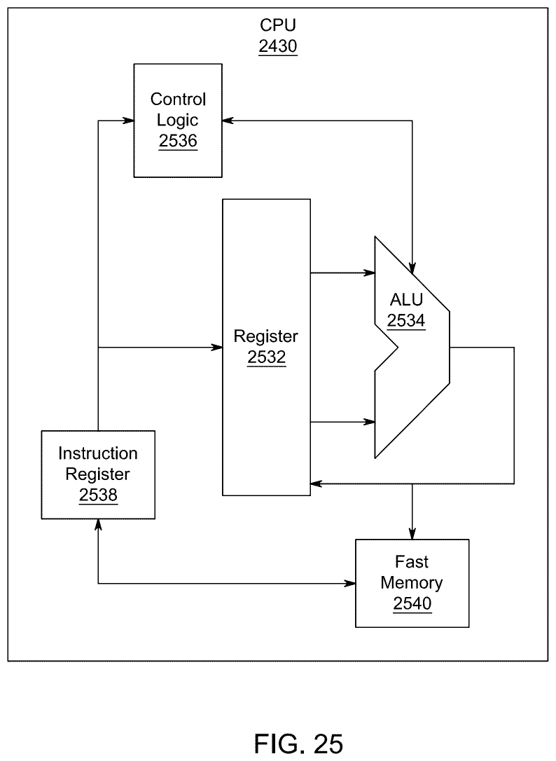

For example, FIG. 25 shows one aspects of the present disclosure of CPU 2430. In one aspects of the present disclosure, the instruction register 2538 retrieves instructions from the fast memory 2540. At least part of these instructions is fetched from the instruction register 2538 by the control logic 1336 and interpreted according to the instruction set architecture of the CPU 2430. Part of the instructions can also be directed to the register 2532. In one aspects of the present disclosure the instructions are decoded according to a hardwired method, and in another aspect of the present disclosure the instructions are decoded according to a microprogram that translates instructions into sets of CPU configuration signals that are applied sequentially over multiple clock pulses. After fetching and decoding the instructions, the instructions are executed using the arithmetic logic unit (ALU) 2534 that loads values from the register 2532 and performs logical and mathematical operations on the loaded values according to the instructions. The results from these operations can be feedback into the register and/or stored in the fast memory 2540. According to certain aspects of the present disclosures, the instruction set architecture of the CPU 2430 can use a reduced instruction set architecture, a complex instruction set architecture, a vector processor architecture, a very large instruction word architecture. Furthermore, the CPU 2430 can be based on the Von Neuman model or the Harvard model. The CPU 2430 can be a digital signal processor, an FPGA, an ASIC, a PLA, a PLD, or a CPLD. Further, the CPU 2430 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 24, the data processing system 2400 can include that the SB/ICH 2420 is coupled through a system bus to an I/O Bus, a read only memory (ROM) 2456, universal serial bus (USB) port 2464, a flash binary input/output system (BIOS) 2468, and a graphics controller 2458. PCI/PCIe devices can also be coupled to SB/ICH 2420 through a PCI bus 2462.

The PCI devices may include, for example, Ethernet adapters, add-in cards, and PC cards for notebook computers. The Hard disk drive 2460 and CD-ROM 2456 can use, for example, an integrated drive electronics (IDE) or serial advanced technology attachment (SATA) interface. In one aspects of the present disclosure the I/O bus can include a super I/O (SIO) device.

Further, the hard disk drive (HDD) 2460 and optical drive 2466 can also be coupled to the SB/ICH 2420 through a system bus. In one aspects of the present disclosure, a keyboard 2470, a mouse 2472, a parallel port 2478, and a serial port 2476 can be connected to the system bus through the I/O bus. Other peripherals and devices that can be connected to the SB/ICH 2420 using a mass storage controller such as SATA or PATA, an Ethernet port, an ISA bus, an LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

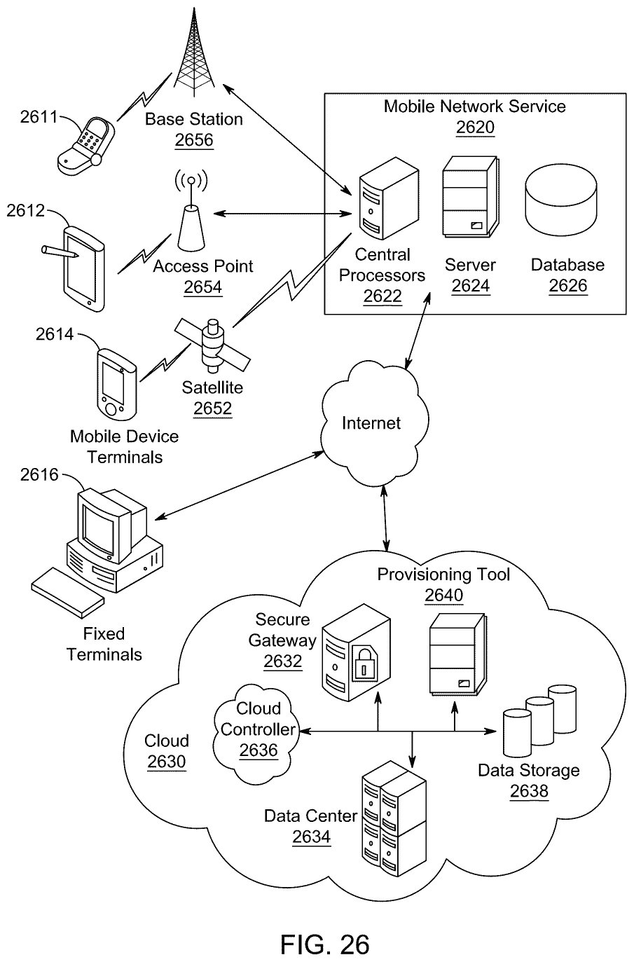

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 26, in addition to various human interface and communication devices (e.g., display monitors, smart phones, tablets, personal digital assistants (PDAs)). More specifically, FIG. 26 illustrates client devices including a smart phone 2611, a tablet 2612, a mobile device terminal 2614 and fixed terminals 2616. These client devices may be commutatively coupled with a mobile network service 2620 via base station 2656, access point 2654, satellite 2652 or via an internet connection. Mobile network service 2620 may comprise central processors 2622, a server 2624 and a database 2626. Fixed terminals 2616 and mobile network service 2620 may be commutatively coupled via an internet connection to functions in cloud 2630 that may comprise security gateway 2632, data center 2634, cloud controller 2636, data storage 2638 and provisioning tool 2640. The network may be a private network, such as a LAN or WAN, or may be a public network, such as the Internet. Input to the system may be received via direct user input and received remotely either in real-time or as a batch process. Additionally, some aspects of the present disclosures may be performed on modules or hardware not identical to those described. Accordingly, other aspects of the present disclosures are within the scope that may be claimed.

The above-described hardware description is a non-limiting example of corresponding structure for performing the functionality described herein.

Numerous modifications and variations of the present disclosure are possible in light of the above teachings. It is therefore to be understood that within the scope of the appended claims, the disclosure may be practiced otherwise than as specifically described herein.

1. A method of controlling a robotic arm based on brain activities, comprising:
measuring a hyperbaric oxygen (HbO) level of a non-disabled subject at target brain areas during a wrist movement using a functional near-infrared spectroscopy (fNIRS) device having light sources and detectors;
detecting one or more brain activities of the non-disabled subject through said detectors based on the HbO level of the non-disabled subject during the wrist movement;
classifying the one or more brain activities corresponding to the wrist movement using one or more classification algorithms and generating a training data set;
generating one or more control signals based on the one or more brain activities for the robotic arm to perform the wrist movement;
detecting one or more brain activities of a disabled subject at the target brain areas based on the HbO level using the fNIRS device;
analyzing the one or more brain activities of the disabled subject based on the training data set; and
generating the one or more control signal for the robotic arm to perform the wrist movement based on the analyzed brain activity of the disabled subject.

2. The method of claim 1, wherein the wrist movement includes at least one selected from the group consisting of wrist extension, wrist flexion, ulnar deviation, and radial deviation of the non-disabled subject.

3. The method of claim 1, wherein the one or more classification algorithms is an Artificial Neural Network (ANN), K-Nearest Neighbor (KNN) or Support Vector Machine (SVM).

4. The method of claim 1, wherein the fNIRS device includes eight light sources and eight detectors.

5. The method of claim 1, wherein the light sources emit light at a first peak emission of 760±10 nm and at a second peak emission of 850±10 nm.

6. A robotic arm to execute the method of claim 1, comprising:
a pin finger, a first finger, and a proximal finger connected together with a joint pin in a palm section of the robotic arm, wherein the palm section comprises a palm;
a wrist connector and a hand connector connecting the palm section to a forearm section through a wrist joint; and
wherein the forearm section comprises an actuator base mounted on a circuit holder to control a movement of the robotic arm.

7. The robotic arm of claim 6, wherein the robotic arm is configured to perform four wrist movements including wrist extension, wrist flexion, ulnar deviation, and radial deviation.

8. A system of provisioning control of a robotic arm based on brain activities, comprising:
a robotic arm;
a functional near-infrared spectroscopy (fNIRS) device with one or more light sources and one or more detectors for measuring a hyperbaric oxygen (HbO) level of at least one non-disabled subject at target brain areas during a wrist movement; wherein the one or more detectors detects one or more brain activities of the non-disabled subject based on the HbO level of the non-disabled subject during the wrist movement;
a classifying means for classifying the one or more brain activities corresponding to wrist movement using one or more classification algorithms and generating a training data set;
a brain-control interface (BCI) generates one or more control signals based on the classified brain activities for the robotic arm to perform the wrist movement;
a detecting means for detecting one or more brain activities of a disabled subject at the target brain areas based on the HbO level using the fNIRS device; and
an analyzing means for analyzing the one or more brain activities of the disabled subject based on the training data set;
wherein, the BCI generates the control signal for the robotic arm to perform the wrist movement based on the analyzed brain activity of the disabled subject.

9. The system of claim 8, wherein the wrist movement includes at least one selected from the group consisting of wrist extension, wrist flexion, ulnar deviation, and radial deviation of the non-disabled subject.

10. The system of claim 8, wherein the one or more classification algorithms an Artificial Neural Network (ANN), K-Nearest Neighbor (KNN) or Support Vector Machine (SVM).

11. The system of claim 8, wherein the fNIRS device includes eight light sources and eight detectors.

12. The system of claim 8, wherein the light sources emit light at a first peak emission of 760±10 nm and at a second peak emission of 850±10 nm.

13. The robotic arm in the system of claim 8, further comprising:
a pin finger, a first finger, and a proximal finger connected together with a joint pin in a palm section of the robotic arm, wherein the palm section comprises a palm;
a wrist connector and a hand connector connecting the palm section to a forearm section through a wrist joint; and
wherein the forearm section comprises an actuator base mounted on a circuit holder to control a movement of the robotic arm.

14. The robotic arm of claim 13, wherein the robotic arm is configured to perform four wrist movements including wrist extension, wrist flexion, ulnar deviation, and radial deviation.
